(19) United States
(12) Patent Application Publication
(10) Pub. No.: US 2005/0085740 A1
(43) Pub. Date: Apr. 21, 2005

(54) Non-invasive breath analysis using field asymmetric ion mobility spectrometry

(76) Inventors: Cristina E. Davis, Cambridge, MA (US); Jeffrey T. Borenstein, Holliston, MA (US); Angela M. Zapata, Arlington, MA (US); Jeffrey A. Gelfand, Cambridge, MA (US); Michael V. Callahan, Cambridge, MA (US); Thomas Osborne Stair, Newton, MA (US); Raanan A. Miller, Boston, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/817,455

(22) Filed: Apr. 1, 2004

Related U.S. Application Data
(60) Provisional application No. 60/459,424, filed on Apr. 1, 2003.

(51) Int. Cl. A61B 5/08

(52) U.S. Cl. 600/532; 422/84

(57) ABSTRACT

An asymmetric field ion mobility spectrometer for breath analysis and a system for analysis of a sample of breath taken from a patient.

### RELATED APPLICATIONS

This application claims the benefit of and priority to provisional patent application Ser. No. 60/459,424 filed in the United States Patent Office on Apr. 1, 2004, the entire contents of which are incorporated by reference herein.

### BACKGROUND

The present invention relates generally to medical diagnostics and more particularly to breath analysis.

Previous detection of chemical agents and biological agents was accomplished with conventional mass spectrometers, time of flight ion mobility spectrometers and conventionally machined FAIM spectrometers.

Conventional mass spectrometers are very sensitive, highly selective and provide a fast response time, but they are also large, expensive, and require significant amounts of power to operate. Also, a conventional mass spectrometer requires a powerful vacuum pump to maintain high vacuum required to isolate ions from neutral molecules and permit detection of selected ions.

Another spectrometric technique is time of flight ion mobility spectrometry, which is a method implemented in portable chemical weapons and explosives detectors. Time of flight ion detection is based not solely on mass, but on charge and cross-section of the molecule as well. However, because of these different characteristics, molecular species identification is not as conclusive and accurate as with mass spectrometry. When time of flight ion mobility spectrometers are reduced in size, for example, to include a drift tube length less than 2 inches, they typically have unacceptable resolution and sensitivity limitations. In time of flight ion mobility, resolution is proportional to the length of the drift tube, with a longer drift tube providing better resolution, as long as the drift tube is also wide enough to prevent all ions from being lost to the side walls due to diffusion. Thus, fundamentally, miniaturization of time of flight ion mobility systems leads to a degradation in system performance.

While conventional time of flight devices are relatively inexpensive and reliable, they suffer from several limitations. First, the sample volume through the detector is small, so to increase spectrometer sensitivity either the detector electronics must have extremely high sensitivity, requiring expensive electronics, or a concentrator is required, adding to system complexity. In addition, a gate and gating electronics are usually needed to control the injection of ions into the drift tube.

FAIM spectrometry was developed in the former Soviet Union in the 1980's. FAIM spectrometry allows a selected ion to pass through a filter while blocking the passage of undesirable ions. One prior FAIM spectrometer was large and expensive, e.g., the entire device was nearly a cubic foot in size and cost over $25,000. These systems are not suitable for use in applications requiring small detectors. They are also relatively slow, taking as much as one minute to produce a complete spectrum of the sample gas, are difficult to manufacture and are not mass producible.

Moreover, the pumps required to draw a sample medium into the spectrometer and to provide a carrier gas can be rather large and consume large amounts of power.

### BRIEF SUMMARY OF THE INVENTION

In one aspect, the invention features a system for analysis of a sample of breath taken from a patient. According to this aspect, a breath sample is provided to an asymmetric field ion mobility spectrometer. The spectrometer includes an ionization source for ionizing the breath sample and creating ions, an analytical gap enclosed by a housing, and an ion filter disposed in the analytical gap downstream from the ionization source. Electrodes are included on an inside surface of the housing, the electrodes are capable of creating an asymmetric electric field to filter the ions. An ion flow generator includes electrodes proximate, but insulated with respect to the ion filter electrodes. The ion flow generator creates an electric field transverse to the asymmetric electric field that propels ions through the asymmetric electric field. An ion detector senses ions not filtered by the ion filter.

In one embodiment, the breath sample is introduced into the spectrometer. Optionally, the patient or subject providing the breath sample breathes according to a standard protocol, for example, prior to providing the breath sample. The breath sample is introduced at, for example, a constant rate or at a fixed volume. The breath sample may be separated according to an intermediate analytical separation technique, by, for example, a classic analytical chemistry separation technique, prior to being introduced into the spectrometer.

In one embodiment, the breath sample is provided at a constant rate of breath expiration, optionally, the breath sample is exhaled into the spectrometer. Alternatively, the breath sample is provided into a channel adapted to introduce the exhaled breath sample into the spectrometer. In another embodiment, the breath sample is contained in a collection vessel, optionally, a conduit is adapted to introduce the breath sample into the spectrometer from the collection vessel. In one embodiment, the system for breath analysis further includes one or more pressure source for introducing the breath sample into the spectrometer.

In yet another embodiment, a user is provided a signal regarding, for example, the rate or volume of the breath sample.

In still another embodiment, the patient providing the breath sample is suspected to have at least pulmonary infection, metabolic disease, chronic progressive degenerative pulmonary disease, lung cancer, or organ dysfunction. In one embodiment, the patient providing the breath sample is receiving a course of drug therapy. In another embodiment, the patient providing the breath sample is suspected to have been exposed to industrial chemicals.

In another embodiment, the ion detector is adapted to sense ions indicative of at least nitric oxide level, pulmonary infection, pulmonary inflammation, metabolic disease, chronic progressive degenerative pulmonary disease, lung cancer, organ dysfunction, industrial chemical exposure. In yet another embodiment, the ion detector is adapted to sense ions of biomarkers indicative of response to drug therapy. The ion detector is adapted to sense ions indicative of at least bacterial infection, viral infection, fungal infection, yeast infection, infectious disease agents, response to biowarfare agents or emerging infectious disease agents.

The spectrometer may be hand held and optionally, has an independent power supply. The spectrometer may be suitable to use in the field, for example, in a remote location.

In another embodiment, the system further includes a data collector to collect data regarding the ion sensed by the ion detector. Such collected data may be evaluated for, for example, a pattern. The collected data and the data patterns provide, for example, analysis, identification and detection information. In one embodiment, the data collector is a personal data assistant that is disposed on the spectrometer.

In yet another aspect, the invention features an asymmetric field ion mobility spectrometer for breath analysis. The spectrometer includes an ionization source for ionizing a breath sample and creating ions, an analytical gap enclosed by a housing, and an ion filter disposed in the analytical gap downstream from the ionization source. Electrodes are included on an inside surface of the housing, and the electrodes are capable of creating an asymmetric electric field to filter the ions. An ion flow generator includes electrodes proximate, but insulated with respect to the ion filter electrodes. The ion flow generator creates an electric field transverse to the asymmetric electric field that propels ions through the asymmetric electric field. An ion detector senses ions not filtered by the ion filter.

In yet another aspect, the invention features an asymmetric filed ion mobility spectrometer for breath analysis. The spectrometer includes an ionization source for ionizing a breath sample and creating ions, an analytical gap, and an ion filter disposed in the analytical gap downstream from the ionization source. The ion filter includes a pair of spaced electrodes for creating an asymmetric electric field to filter the ions. An ion flow generator includes a plurality of spaced discrete electrodes insulated from the pair of spaced electrodes for creating an electric field transverse to the asymmetric electric field for propelling ions through the asymmetric electric field. An ion detector senses ions not filtered by the ion filter.

In yet another aspect, the invention features an asymmetric field ion mobility apparatus for identification of ion species in a breath sample. The apparatus includes an ionization source for ionizing a breath sample and creating ions and an ion filter disposed in a flow path. The flow path includes a longitudinal axis for the flow of ions and the filter, which is compensated, supplies an asymmetric filter field transverse to the longitudinal axis. The ion flow generator is adapted to longitudinally propel ions along the flow path in the compensated asymmetric filter field. The ion filter passes a species of propelled ions that have a set of characteristics correlated with the compensation and the correlation aids or facilitates in the identification of this species.

One or more aspects of the invention may provide one or more of the following advantages. A FAIM spectrometer may more quickly and accurately control the flow of a gas sample and produce sample spectrum then conventional analysis devices. The gas sample that is provided to the FAIM spectrometer is, for example, a breath sample taken from a patient or a test subject. FAIM spectrometry has sensitive detection limits that may enable detection of compounds in breath and determination of distinctions between compounds in breath that are unable to be resolved by other analytical techniques.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1A is a schematic block diagram of a FAIM spectrometer.

FIG. 1B is a schematic representation of ions as they pass through a FAIM spectrometer.

FIG. 1C is a three dimensional view of a packaged micromachined FAIM spectrometer.

FIG. 2 is a schematic representation of hypothetical migrating ion species.

FIG. 3A is a schematic representation of a FAIM spectrometer chip.

FIG. 3B is a prototype micromachined FAIM spectrometer.

FIG. 4 is a schematic flow diagram of an embodiment of a system for breath analysis.

FIG. 5A shows a flame ionization detector (FID) instrument response as a function of compound concentration for a homogenous ketone mixture.

FIG. 5B shows a FAIM spectrometer response as a function of compound concentration for a homogenous ketone mixture.

FIG. 6A shows a chromatogram of a FAIM spectrometer coupled to a GC column.

FIG. 6B shows a chromatogram of a standard GC output.

FIG. 7A shows a chromatogram of a standard GC output of co-eluted Acetone and Butanone species.

FIG. 7B shows a GC-FAIM spectra of co-eluted Acetone and Butanone species.

FIGS. 8A and 8B respectively show a comparison of FID chromatography and FAIM spectrometry average responses for a variety of homogeneous alcohol mixtures.

FIG. 9A shows measurement of two chemical isomers, para- and meta-xylene, with Time of Flight/lon Mobility Spectrometry (TOF-IMS).

FIG. 9B shows measurement of two chemical isomers, para- and meta-xylene, with the FAIM spectrometer.

FIG. 10 is a schematic flow diagram of an embodiment of a system for breath analysis.

FIG. 11 is a schematic flow diagram of an embodiment of a system for breath analysis.

FIG. 12A is a schematic flow diagram of an embodiment of FAIM spectrometry analysis.

FIG. 12B is a schematic flow diagram of an embodiment of FAIM spectrometry analysis.

FIG. 13 is a schematic flow diagram of an embodiment of a system for breath analysis.

FIG. 14 is a schematic flow diagram of an embodiment of breath sample analysis.

FIG. 15 shows a FAIM spectra data plot of intensity versus compensation voltage as a function of retention time for pentanone.

FIGS. 16A-16Q show elements of breath analysis systems and breath analysis systems featuring various combinations of elements.

FIG. 17 shows spectra results from pyrolysis-mass spectrometry.

FIG. 18A shows spectra results from pyrolysis-FAIM spectrometry for Picolinic acid (PA).

FIG. 18B shows spectra results from pyrolysis-FAIM spectrometry for Dipicolinic acid (DPA).

FIG. 19A show gas chromatography/FAIM spectrometry spectra from breath samples taken from one subject.

FIG. 19B show gas chromatography/FAIM spectrometry spectra from breath samples taken from another subject.

FIG. 20A shows FAIM spectrometer spectra including peak responses for low ppm NO₂ concentrations.

FIG. 20B shows FAIM spectrometer spectra including peak responses for low ppm NO concentrations.

FIG. 20C shows a spectra plot with concentration of NO on the x-axis and peak height on the y-axis.

FIG. 20D shows a spectra plot with concentration of NO on the x-axis and peak area on the y-axis.

### DETAILED DESCRIPTION

The following patents and applications are incorporated by reference herein in their entirety: U.S. Pat. No. 6,512,224 entitled “Longitudinal Field Driven Asymmetric Ion Mobility Filter and Detection System,” U.S. Pat. No. 6,495,823 entitled “Micromachined Field Asymmetric Ion Mobility Filter and Detection System,” U.S. patent application Ser. No. 10/082,803 entitled “Longitudinal Field Driven Ion Mobility Filter and Detection System,” and U.S. patent application Ser. No. 10/697,708 entitled “High Field Ion Mobility Method and Apparatus for Detection of Biomarkers.”

1. FAIM Spectrometer

FAIM spectrometers operate at ambient temperature and pressure. A micromachined FAIM spectrometer has been developed as a portable unit that is mobile and hand-held. The spectrometer produces spectra that differentiates between compounds that co-elute in gas chromatography/mass spectrometry (GC-MS), often yielding an improved ability to identify samples. For matrix-assisted laser desorption ionization/mass spectrometery (MALDI-MS), a statistical model has demonstrated the ability to distinguish between roughly 10 species similar to B. subtilis when the spectral masses are grouped in 1.5 Daltons (Da) ranges. This is due to roughly the same number of proteins per unit-mass interval. Recent data also suggests a 75% correct identification rate using MALDI-MS with no false positives. However, with the FAIM spectrometer technology, even larger numbers of species may easily be distinguished, as the spectra may be more easily deconvoluted than those of MS due to differing ion mobilities.

FAIM spectrometers, including Micromachined FAIM spectrometers, may be employed as detectors for chemical and biological sensing applications. FAIM spectrometers are also referred to as Differential Mobility Spectrometers (DMS). FAIM spectrometers are quantitative and have extremely sensitive detection limits, down to the parts-per-trillion range. The FAIM spectrometry method uses the non-linear mobility dependence of ions on high strength RF electric fields for ion filtering, and operates in air at atmospheric pressure. This method enables the rapid detection and identification of compounds that cannot be resolved by other analytical techniques. FAIM spectrometers scale down well, allowing miniaturization of the analytical cell using MicroElectroMechanical (MEMS) fabrication, while preserving sensitivity and resolution. These and other advantages of FAIM spectrometry make it attractive as a quantitative detector that is sufficiently low in cost to be practical for use in the field, for example, in point-of-care diagnostics in clinical settings.

Conceptually, the operating principle of the FAIM spectrometer is similar to that of a quadrupole mass spectrometer, with the significant distinction that it operates at atmospheric pressure so it measures ion mobility rather than ion mass. Mobility is a measure of how easily an ion travels through the air in response to an applied force, and is dependent on the size, charge and mass of the ion. A FAIM spectrometer acts as a tunable ion filter.

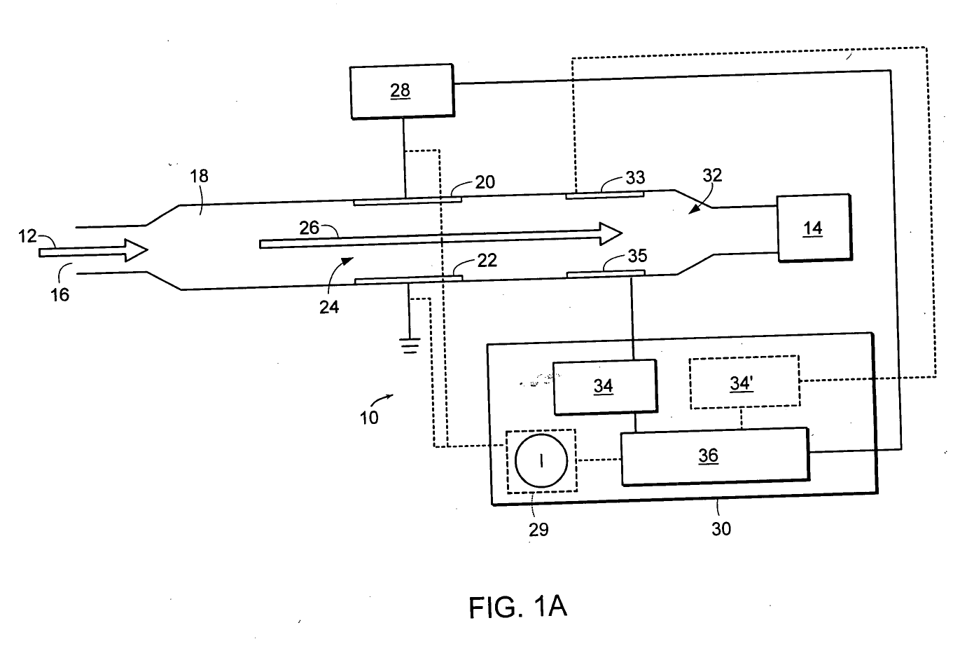

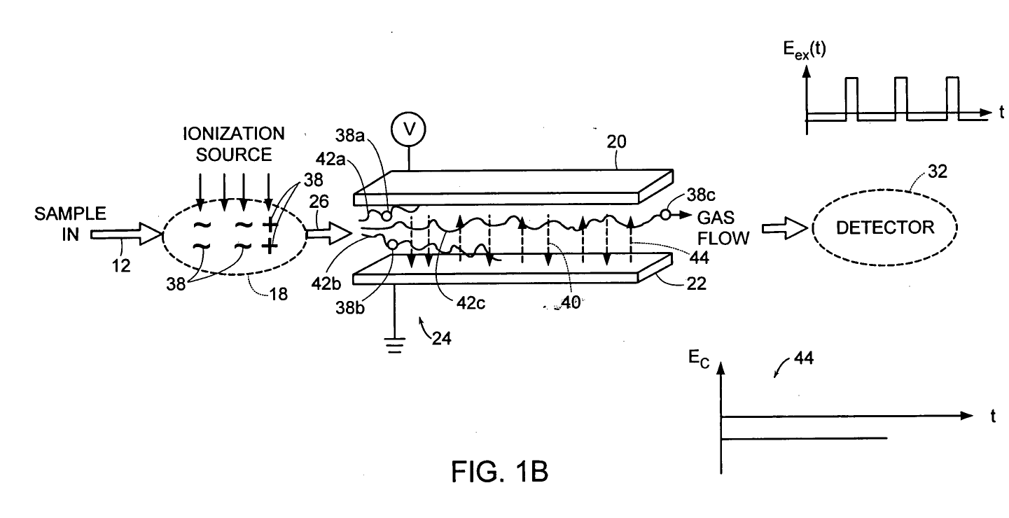

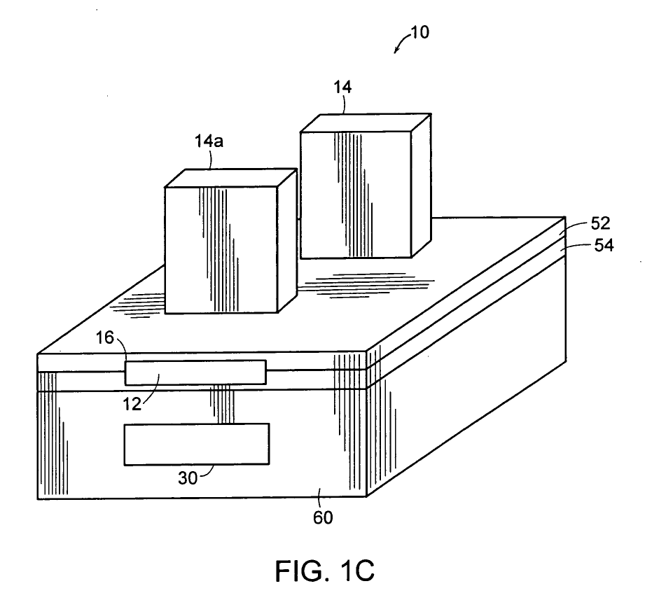

To perform a measurement, a gas sample is introduced into the spectrometer, where it is ionized, and the ions are transported through an ion filter towards the detecting electrodes (Faraday plates) by a carrier gas, as shown in FIGS. 1A and 1B. The FAIM spectrometer can separate chemical components of a substance based on differing ion mobilities.

As shown in FIGS. 1A and 1B, the FAIM spectrometer operates by introducing a gas, indicated by arrow 12, into ionization region 18. The ionized gas follow flow path 26 and are passed between parallel electrode plates 20 and 22 that make up the ion filter 24. As the gas ions pass between plates 20 and 22, they are exposed to an electric field between electrode plates 20 and 22 induced by a voltage applied to the plates. In one embodiment, the electric field produced is asymmetric and oscillates in time.

As ions pass through filter 24, some are neutralized by plates 20 and 22 while others pass through and are sensed by ion detector 32. In one embodiment, the detector 32 includes a top electrode 33 at a predetermined voltage and a bottom electrode 35, typically at ground. The top electrode 33 deflects ions downward to the bottom electrode 35. However, either electrode may detect ions depending on the ion and the voltage applied to the electrodes. Moreover, multiple ions may be detected by using top electrode 33 as one detector and bottom electrode 35 as a second detector.

The electronic controller 30 may include, for example, an amplifier 34 and a microprocessor 36. Amplifier 34 amplifies the output of detector 32, which is a function of the charge collected by electrode 35 and provides the output to microprocessor 36 for analysis. Similarly, amplifier 34′, shown in phantom, may be provided where electrode 33 is also utilized as a detector.

Referring now to FIG. 1B, as ions 38 pass through alternating asymmetric electric field 40, which is transverse to gas flow 12, electric field 40 causes the ions to “wiggle” along paths 42a, 42b and 42c. Time varying voltage V is typically in the range of +/−(1000-2000) volts and creates electric field 40 with a maximum field strength of 40,000 V/cm. The path taken by a particular ion is a function of its mass, size, cross-section and charge. Once an ion reaches electrode 20 or 22, it is neutralized. A second, bias or compensation field 44, typically in the range of +/−2000 V/cm due to a +/−100 volt dc voltage, is concurrently induced between electrodes 20 and 22 by a bias voltage applied to plates 20 and 22, also by voltage generator 28, FIG. 1A, in response to microprocessor 36 to enable a preselected ion species to pass through filter 24 to detector 32. Compensation field 44 is a constant bias that offsets alternating asymmetric field 40 to allow the preselected ions, such as ion 38c to pass to detector 32. Thus, with the proper bias voltage, a particular species of ion will follow path 42c while undesirable ions will follow paths 42a and 42b to be neutralized as they encounter electrode plates 20 and 22.

The output of FAIM spectrometer 10 is a measure of the amount of charge on detector 32 for a given bias electric field 44. The longer the filter 24 is set at a given compensation bias voltage, the more charge will accumulate on detector 32. However, by sweeping compensation voltage 44 over a predetermined voltage range, a complete spectrum for sample gas 12 can be achieved. The FAIM spectrometer according to the present invention requires typically less than thirty seconds and as little as one second to produce a complete spectrum for a given gas sample. By varying compensation bias voltage 44, the species to be detected can be varied to provide a complete spectrum of the gas sample.

The FAIM spectrometer may include an ion flow generator for propelling the ions 38 generated by the ionization source through the asymmetric electric field 40 created by the ion filter 24 and toward the detector 32. Opposed electrode pairs may create the ion flow generator, for example ring electrode pairs and/or planar electrode pairs. Also, the ion flow generator may create a longitudinal electric field in the direction of the intended ion travel, toward, for example, the detector 32. The strength of the longitudinal electric field can be constant in time or space and can vary with time and space. The longitudinal electric field can propel ions 38 through asymmetric electric field 40.

In one embodiment, the ion filter 24 is disposed in an analytical gap, downstream from the ionization source, for creating an asymmetric electric field to filter ions generated by the ionization source.

FAIM spectrometers are disclosed in greater detail in U.S. Pat. No. 6,512,224 entitled “Longitudinal Field Driven Asymmetric Ion Mobility Filter and Detection System,” U.S. Pat. No. 6,495,823 entitled “Micromachined Field Asymmetric Ion Mobility Filter and Detection System,” U.S. patent application Ser. No. 10/082,803 entitled “Longitudinal Field Driven Ion Mobility Filter and Detection System,” which are incorporated by reference herein.

Packaged FAIM spectrometer 10, FIG. 1C, may be reduced in size, for example a reduced size FAIM spectrometer measures one inch by one inch by one inch. Optionally, a pump 14 is mounted on substrate 52 for drawing a gas sample 12 into inlet 16. A recirculation pump 14a may optionally be employed to introduce clean dry air into flow path 26, FIG. 1A, prior to or after ionization of the gas sample. Electronic controller 30 may be etched into silicon control layer 60, which combines with substrates 52 and 54 to form a housing for spectrometer 10. Substrates 52 and 54 and control layer 60 may be bonded together by, for example, using anodic bonding, to provide an extremely small FAIM spectrometer. Micro pumps 14 and 14a provide a high volume throughput to further expedite the analysis of gas sample 12. Pumps 14 and 14a may be, for example, conventional miniature disk drive motors fitted with small centrifugal air compressor rotors or micromachined pumps, which produce flow rates of 1 to 4 liters per minute. One example of pump 14 is available from Sensidyne, Inc., Clearwater, Fla.

Generally, the ion filter is tuned by adjusting the electric fields applied between the parallel ion filter electrodes. Two fields are applied; an asymmetric waveform electric field which alternates between a high strength and low strength field, and a low strength DC compensation electric field. The amplitude of the asymmetric field is kept constant, while the compensation voltage (compensation electric field) levels are adjusted to select the particular ion species allowed to pass through the ion filter. Once the selected ion species passes the ion filter electrodes, it is detected as an ion current upon collision with the detector electrodes. Depending on the electric field conditions applied to the FAIM spectrometer, ion species are selected and permitted to pass through the ion filter region to be collected at a detector, which may be, for example, a simple charge collector electrode. Unwanted (i.e., uncompensated) ions are scattered towards the ion filter electrodes, neutralized, and swept out by the carrier gas. The filtering mechanism is governed by the interaction between the ion and the net applied field, which alternates between high and low electric field strengths.

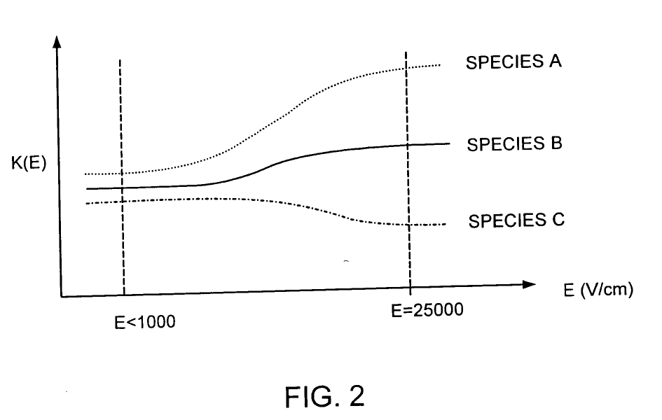

The particular compensation voltage required to select an ion species to pass through the filter is governed by its differential mobility. The mobility of an ion in air is field-dependent, and can change significantly as the field strength increases. The compensation voltage required to allow a particular ion to pass through the filter exploits this field/mobility relationship. FIG. 2 shows hypothetical ion species that migrate in a high strength electric field. In FIG. 2 the x-axis shows increasing field strength in voltage per centimeters and the y-axis shows kinetic energy in, for example, Joules. The different ion species, species A, species B and species C, migrate differently. In a low electric field the differential mobility of species A, B, and C is small. As the field strength is increased, each species acquires different ion mobility. The differences in ion mobility provide a basis for separation and detection. The electric field conditions required to permit a particular ion to penetrate though the filter to the detector are specific to each ion species. By noting the applied field conditions, or voltages, and the current level at the detector electrode, various ions species can be identified.

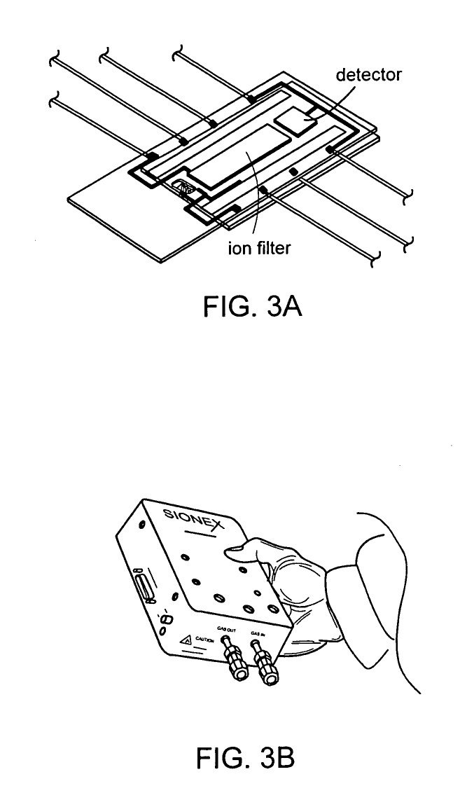

FIG. 3A shows a schematic of a FAIM spectrometer chip. Because the FAIM spectrometer functions as a filter, the longer the field conditions are set at one particular value, the more ions are collected at the detector. This improves the signal-to-noise ratio, thus enabling increased sensitivity. Since mass production techniques and batch fabrication methods can be employed in producing this miniaturization, significantly less expensive devices can be manufactured. A prototype development platform designed for proof-of-principle and user evaluation is shown in FIG. 3B. The evaluation prototype was packaged to allow maximum flexibility and troubleshooting. Future systems can be miniaturized further.

FAIM spectrometry has several advantages over conventional ion mobility spectrometers, which are much larger, more expensive, and operate with short pulses of ions. In the FAIM spectrometry analysis, the ions are introduced continuously into the ion filter with nearly 100% throughput of the “tuned” ions reaching the detector. This allows the FAIM spectrometer to have an extremely high sensitivity even though it is significantly smaller. This approach also avoids the complexity of generating short, spatially well-confined, ion pulses required in the conventional IMS (Ion Mobility Spectrometry). In fact, the FAIM spectrometry approach actually benefits from miniaturization, since the electric fields required to filter the ions are on the order of 10,000 V/cm. By reducing the gap dimensions to the order of 500 microns, the voltages required for ion filtering are easily achievable.

FAIM spectrometers may be employed to detect, analyze and/or diagnose conditions impacting patient health and patient exposure to certain chemicals in the environment. Suitable patient biological samples that may be tested by FAIM spectrometry include samples prepared from any tissue, cell, or body fluid. More specifically, biological samples include tissue (e.g., from biopsies), blood, serum, plasma, nipple aspirate, urine, tears, saliva, cells, soft and hard tissues, organs, semen, feces, urine, sputum, pancreatic fluid, bile, lymph, cerebrospinal fluid, pus, amniotic fluid and the like. The biological samples may be obtained from any suitable organism including eukaryotic, prokaryotic, or viral organisms. The biological samples may include biological molecules including macromolecules such as polypeptides, proteins, nucleic acids, enzymes, DNA, RNA, polynucleotides, oligonucleotides, nucleic acids, carbohydrates, oligosaccharides, polysaccharides. The biological samples may include fragments of biological macromolecules set forth above, such as nucleic acid fragments, peptide fragments, and protein fragments. The biological samples may include complexes of biological macromolecules set forth above, such as nucleic acid complexes, protein-DNA complexes, receptor-ligand complexes, enzyme-substrate, enzyme inhibitors, peptide complexes, protein complexes, carbohydrate complexes, and polysaccharide complexes. The biological samples may include small biological molecules such as amino acids, nucleotides, nucleosides, sugars, steroids, lipids, metal ions, drugs, hormones, amides, amines, carboxylic acids, vitamins, coenzymes, alcohols, aldehydes, ketones, fatty acids, porphyrins, carotenoids, plant growth regulators, phosphate esters and nucleoside diphospho-sugars. The biological samples also may include synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents. The biological samples also may include organic compounds, volatile organic compounds, semi-volatile organic compounds, synthetic polymers, oligomers, and copolymers. Any suitable mixture or combination of the substances specifically recited above may also be included in the biological samples.

2. Breath Sample

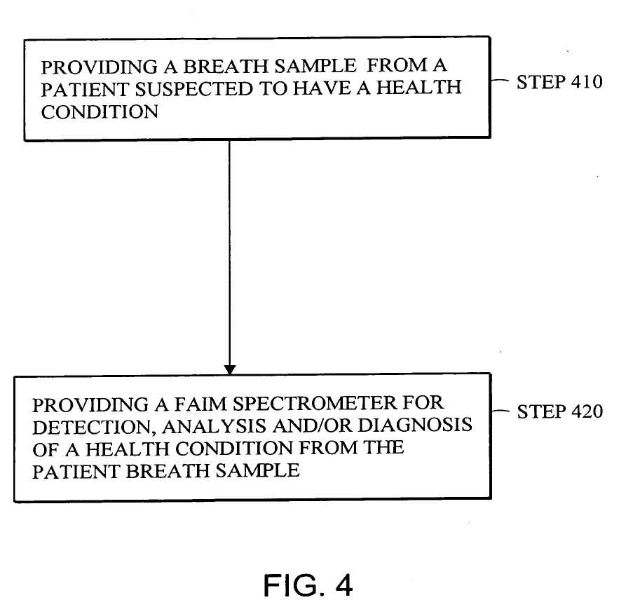

FAIM spectrometers may be employed to analyze breath samples to detect, identify, and diagnose conditions impacting patient health. For example, FAIM spectrometers may be used detect, identify, and diagnose patient exposure to certain chemicals in the environment. A breath sample from, for example, a human patient or human subject is an exemplary patient biological sample that is analyzed by FAIM spectrometry. Referring to FIG. 4, in which a flow diagram depicts an embodiment of a system for breath analysis including providing a breath sample from a patient suspected to have a health condition (STEP 410) and providing a FAIM spectrometer for detection, analysis and/or diagnosis of a health condition from the patient breath sample (STEP 420).

There are dozens of volatile organic compounds in exhaled human breath that show promise for diagnosis and management of diseases, but little technical or clinical research and development has been conducted to date. Some volatile gases are produced by specific disease conditions, and a number of these volatile gases can be smelled by physicians on the patient's breath. Exemplary volatile gasses include ketones in starvation and ketoacidosis, feculent amines in bowel obstruction, and bacterial byproducts in anaerobic infections. Several diagnostic tests measure exhaled hydrogen after a specific sugar or starch load to demonstrate lactose deficiency, malabsorption, bacterial overgrowth of the small bowel, or pancreatic function in cystic fibrosis. One test for Helicobacter pylori requires the patient to first consume ¹³C labeled urea, after which ¹³C labeled CO₂ is detected in exhaled breath. Other labeled metabolites are used in a variety of gastroenterology tests. Exhaled nitric oxide is measured as a non-specific marker of inflammation in the lungs. Testing of exhaled breath has also been proposed as a rapid toxicology test for carbon monoxide and methanol. Two volatile hydrocarbons, ethane and pentane, are produced by the peroxidation of linoleic and linolenic acid, polyunsaturated fatty acids found in cellular membranes that are oxidized during tissue ischemia and reperfusion injury. Breath pentane is elevated in proportion to ischemia and inflammation in heart disease. Breath pentane shows particular promise as a marker for reperfusion injury and could be used to guide the rate of infusion of thrombolytic drugs or the percent of supplemental oxygen.

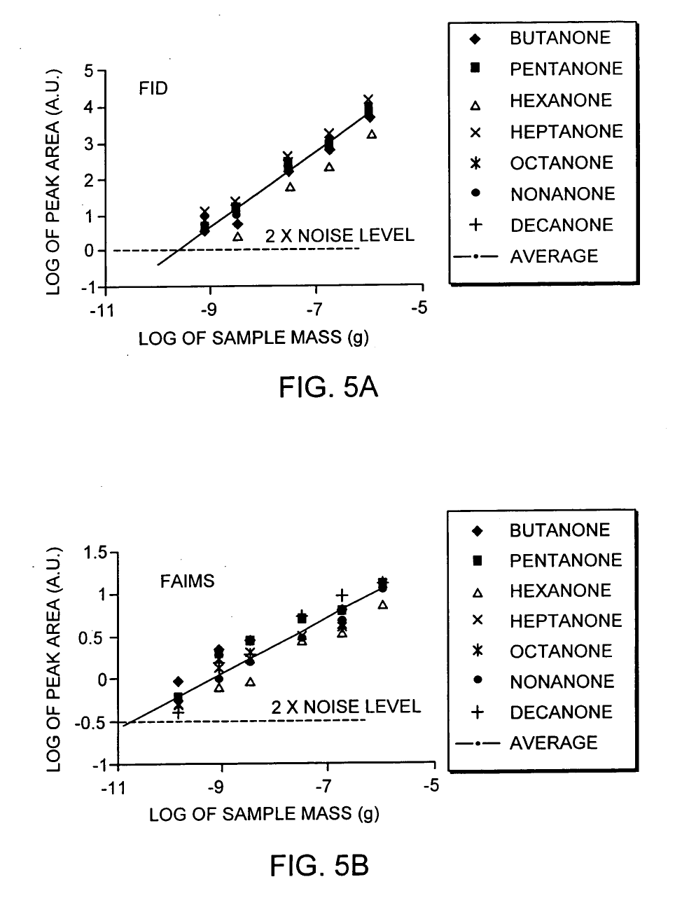

Prior breath studies using gas chromatography, mass spectrometry, or both have been hampered by samples saturated with water vapor, variable ambient levels of gases being measured, ambient pentane dissolved in body fat, and co-elution of isoprene. Such prior issues may not present problems for analysis with FAIM spectroscopy. For example, experiments conducted indicate that average FAIM spectrometer detection limits are approximately an order of magnitude better than those obtained with flame ionization detector (FID). FIGS. 5A and 5B show a FID and a FAIM spectrometer instrument response as a function of compound concentration for a homogenous ketone mixture. The average FID detection limit is 200 picograms (pg), while the FAIM spectrometer limit is 20 pg. Because many disease states are correlated with breath biomarkers, it is expected that unique biomarkers that are within the FAIM spectroscopy detection range will be discovered.

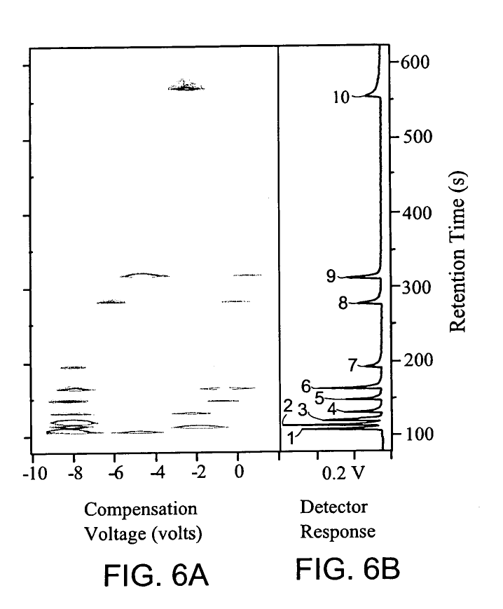

The ion information provided by FAIM spectroscopy offers a second dimension of information to a GC chromatogram and it offers the ability to enhance compound identification. FIG. 6A shows a chromatogram of a FAIM spectrometer coupled to a GC column. FIG. 6B, which is shown opposite FIG. 6A, shows the standard GC output, which is typical of what is seen in FID.

In FIG. 6A, in the spectra from the FAIM spectrometer coupled to a GC column, the chromatogram is the sum of the peak intensities for the product ions created. GC-FAIM spectrometry provides three levels of information: retention time (as shown as the y-axis), compensation voltage (as shown as the x-axis, and ion intensity (is the z-axis, however, the z-axis is not shown in FIG. 6A). The three dimensions provided by GC-FAIM spectrometry provide a means of fingerprinting the compounds eluted from the GC. The spectra are obtained simultaneously for positive and negative ions, eliminating the need of serial analysis under different instrumental conditions, as required in MS. The information provided by GC-FAIM spectrometry will potentially eliminate the need of external calibration through standards.

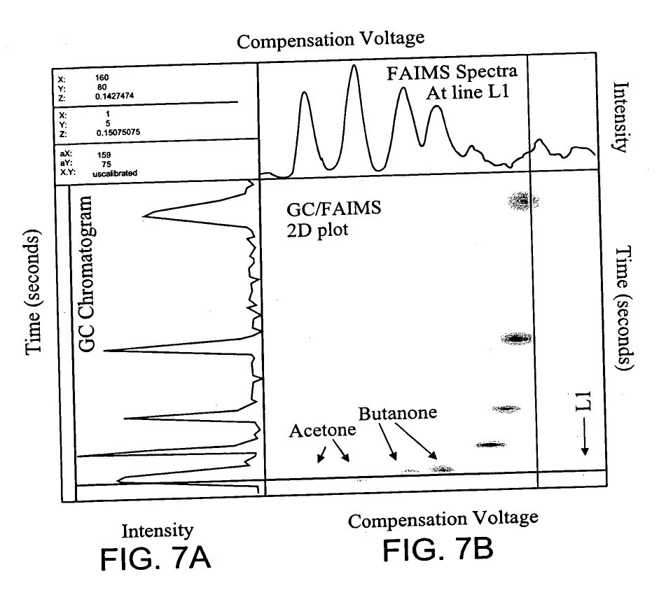

If, during GC column analysis, chromatographic conditions result in co-eluted peaks, the FAIM spectrometer resolves the co-eluted peaks and elucidates compound information. Exemplary chromatographic conditions include fast temperature ramps to reduce analysis time. FIGS. 7A and 7B show Acetone and Butanone that co-eluted during GC column analysis when the chromatographic runtime was decreased. As shown in FIG. 7A the Acetone and Butanone species are not resolved under GC alone, they appear together as a single peak *. However, GC-FAIM spectrometry is able to resolve the co-eluted species into distinct Acetone and Butanone peaks, as shown in FIG. 7B.

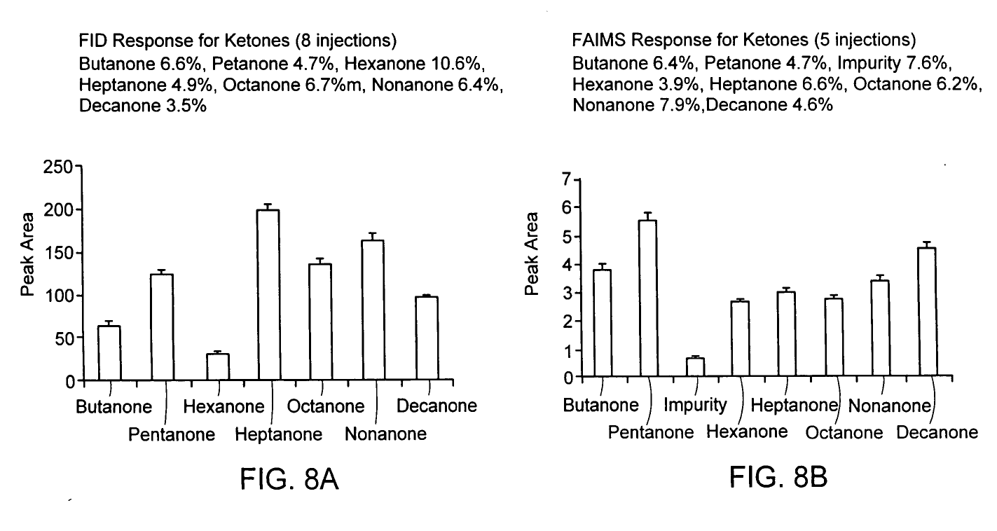

In this way, a fast GC can be used while maintaining required compound resolution. The precision of the FAIM spectrometer compares very well to FID reproducibility. The similarity in precision of FID and FAIM spectrometry is shown by the small deviations illustrated in FIGS. 8A and 8B. FIGS. 8A and 8B respectively show a comparison of FID chromatography and FAIM spectrometry average responses for a variety of homogeneous alcohol mixtures. FIGS. 8A and 8B compare the error bar sizes for the FID and FAIM specrometry methods, respectively.

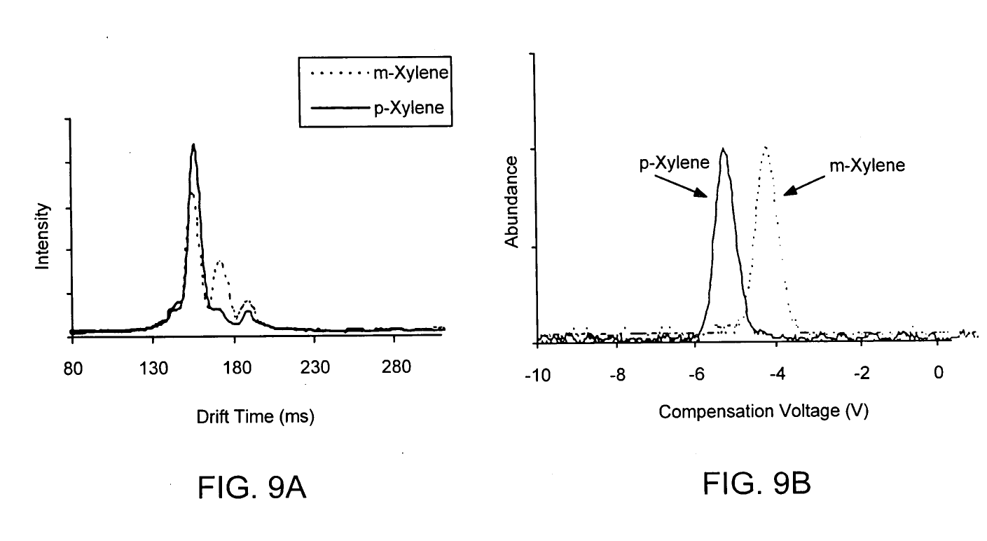

The information provided by the FAIM spectrometer offers the ability to obtain unambiguous compound identification. A striking example is the ability of the FAIM spectrometer to deconvolute closely related species such as isomers. Two isomers of xylene (meta- and para-xylene) were measured using time-of-flight ion mobility spectrometry (TOF-IMS) and FAIM spectrometry. Separation of these two isomers presents a significant challenge for most analytical instruments. The isomers singular molecular weight value precludes analysis by mass spectrometry. Measurement of the two chemical isomers with a conventional TOF-IMS produces overlapping peaks, which are extremely difficult to deconvolute, as shown in FIG. 9A, with the x-axis representing drift time in milliseconds and the y-axis representing intensity in total ion counts (volt output). GC is capable of resolving the peaks, but this process requires in excess of 20 minutes of analysis time. By contrast, the FAIM spectrometer enables clear resolution of the para- and meta-xylene isomers in less than 1 second, as shown in FIG. 9B, with the x-axis representing compensation voltage (V) and the y-axis representing Abundance is the voltage output from the FAIM spectrometer.

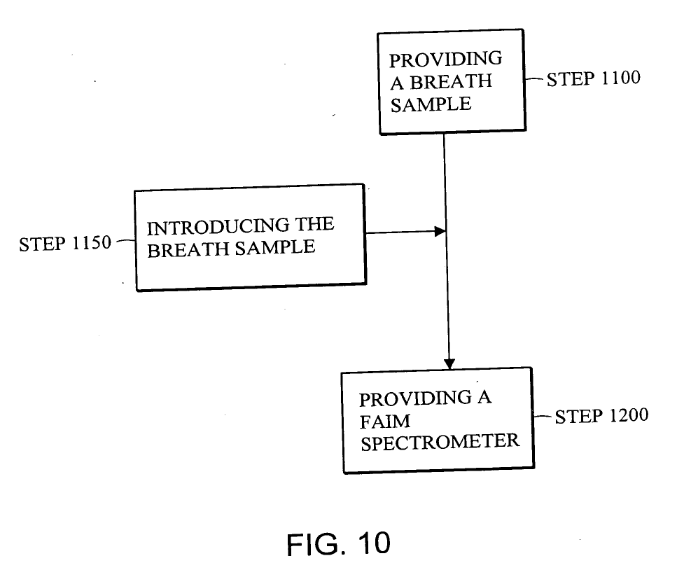

In accordance with at least one aspect of the invention, a breath sample is introduced into the FAIM spectrometer. Referring to FIG. 10, in which a flow diagram depicts an embodiment of a system of the invention including providing a breath sample (STEP 1100) and providing a FAIM spectrometer (STEP 1200). In one embodiment, the breath sample is exhaled directly into the FAIM spectrometer through, for example, the inlet to the gas flow path.

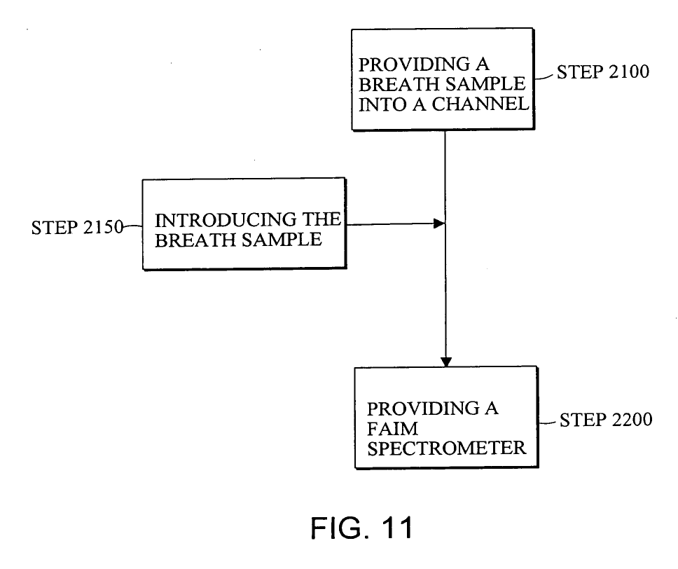

Alternatively, referring to FIG. 11, in which a flow diagram depicts an embodiment of a system of the invention including providing a breath sample into a channel (Step 2100) and providing a FAIM spectrometer (STEP 2200). The breath sample is provided by, for example, a channel connected to the FAIM spectrometer that draws the breath sample into the FAIM spectrometer gas flow path as the breath is exhaled from the patient. Suitable channels include tubes, pipes, facemasks, mouthpieces, ventilators or other channels that are capable of passing the patient breath sample from the patient (STEP 2100) to the FAIM spectrometer (STEP 2200). Suitable channels are manufactured from, for example, inert materials including polymers, copolymers, glass, metals and combinations thereof. Disposable channels may be employed to avoid cross contamination between various patients whose breath is tested using a single FAIM spectrometer. Alternatively, one or more non-disposable channels are affixed to a FAIM spectrometer employed to analyze one or more patient's breath.

Referring still to FIGS. 10 and 11, in some embodiments, after the breath sample is provided an optional step includes introducing the breath sample (STEP 1150 and STEP 2150) to the provided FAIM spectrometer (STEP 1200 and STEP 2200). Upon introduction to the FAIM spectrometer, the breath sample may be analyzed by the FAIM spectrometer.

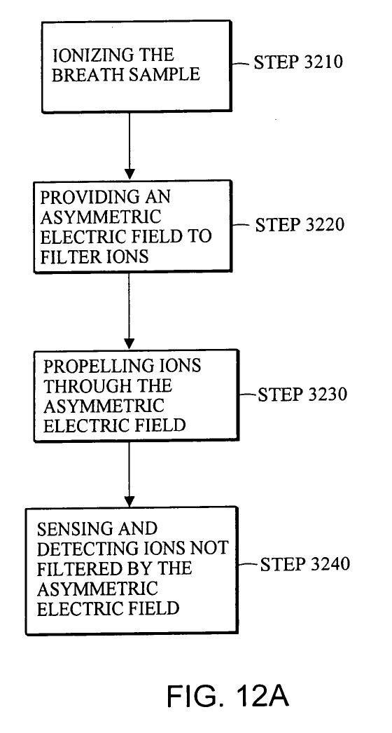

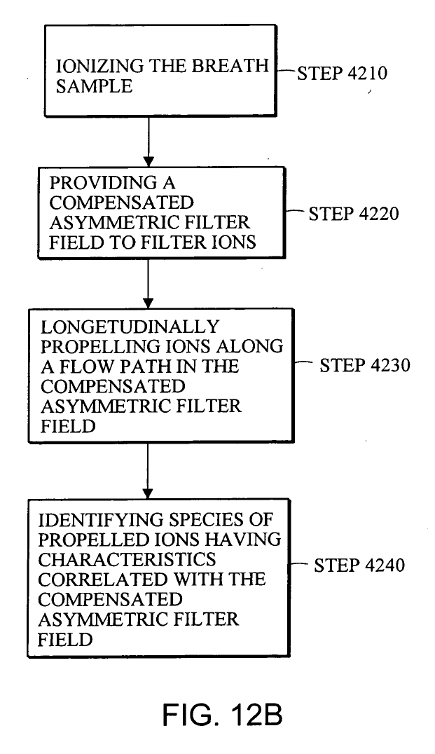

In one aspect of the invention, referring now to FIG. 12A, a flow diagram depicts a FAIM spectrometer of an embodiment of the invention in which the FAIM spectrometer ionizes the breath sample (STEP 3210). Providing an asymmetric electric field to filter ions (STEP 3220) created in the ionizing step. Propelling ions through the asymmetric electric field (STEP 3230) moves ions through the asymmetric electric field. Sensing and detecting ions not filtered by the asymmetric electric field (STEP 3240) analyzes the breath sample ions.

In another aspect of the invention, referring now to FIG. 12B, a flow diagram depicts a FAIM spectrometer of an embodiment of the invention in which the FAIM spectrometer ionizes the breath sample (STEP 4210). Providing a compensated asymmetric filter field to filter ions (STEP 4220) filters ions created in the ionization step. Longitudinally propelling ions along a flow path in the compensated asymmetric filter field (STEP 4230) moves ions thought the compensated asymmetric filter field. Identifying species of propelled ions having characteristics correlated with the compensated asymmetric filter field (STEP 4240) analyzes the breath sample ions.

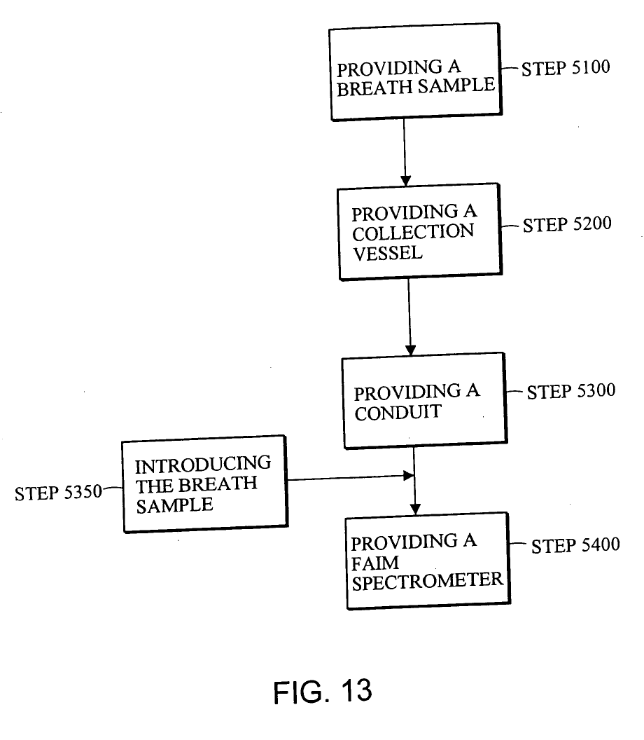

In some embodiments, referring now to FIG. 13, breath samples are stored in collection vessels for later analysis by FAIM spectrometry. A solid phase micro-extraction (SPME) fiber assembly provides a suitable collection vessel. According to this collection method, the SPME fiber is placed in proximity to the mouth of the patient to collect the breath sample. Micromolded polymers capable of absorbing and desorbing chemical constituents in breath may also be employed as collection vessels. Such micromolded polymers are similar to SPME fibers.

Other suitable collection vessels include a closed impermeable container or bag, for example, a balloon that the patient breathes into. Referring still to FIG. 13, a breath sample is provided (STEP 5100) when patients exhale several times into a provided collection vessel (STEP 5200). For example, patients exhale several times into a balloon to inflate the balloon. Thereafter, the balloon is sealed for subsequent analysis via FAIM spectrometry. Suitable balloons include, for example, Mylare or Tedlar® balloons as typically used in party favors and gifts. Suitable collection vessels are made from, for example, glass, metals and polymers and copolymers including nylon, polyester, polyethyleneterephthalate (PET), polyethylene, polyester, vinyl fluoride. Any suitable mixture or combination of such materials may also be employed to make a suitable collection vessel. In one embodiment, the collection vessel is disposable to avoid contamination between patients. In another embodiment, the collection vessel has a port that is accessed to deliver the breath sample.

A conduit is provided (STEP 5300), a FAIM spectrometer is provided (STEP 5400) and the breath sample is introduced into the FAIM spectrometer from the collection vessel through the conduit disposed on, for example, the FAIM spectrometer. A needle or a hose disposed on the FAIM spectrometer provides a suitable conduit for a breath sample to flow from a collection vessel into the FAIM spectrometer. In one embodiment, the conduit is a needle disposed on the FAIM spectrometer that is employed to access the breath sample by piercing a port disposed on the balloon containing a breath sample.

In one embodiment, the pressure from carrier gas present in the FAIM spectrometer introduces and pushes the breath sample through the FAIM spectrometer. In another embodiment, a pump is employed to introduce the breath sample to the FAIM spectrometer. A pump, for example, moves the breath sample from the collection vessel into the FAIM spectrometer via the needle disposed on the FAIM spectrometer. The breath sample may be pushed from the collection vessel into the FAIM spectrometer by pressure. Alternatively, the breath sample is pulled, by suction or vacuum pressure, from the collection vessel into the FAIM spectrometer through a conduit present on the FAIM spectrometer. One or more pumps may be present on, in, or about the FAIM spectrometer, the spectrometer housing, the conduit or the collection vessel. One exemplary pump is located at an end of the system, e.g., at the terminal end of the spectrometer housing, and the pump draws the sample through the system for analysis. In another embodiment, after the breath sample is provided, the breath sample is introduced (STEP 5350) into the provided FAIM spectrometer.

In one embodiment, the breath samples provided are introduced into the FAIM spectrometer and are provided at a constant flow rate. A patient provides constant rate of breath expiration into the FAIM spectrometer through, for example, an inlet on the spectrometer or a channel connected to the spectrometer. Alternatively, a constant rate of breath sample is provided from a collection vessel into the FAIM spectrometer via a conduit disposed on the spectrometer. For example, at least one pump is adapted to move the breath sample from the collection vessel to the spectrometer at a constant flow rate. Such a pump may be present in, on, or about the FAIM spectrometer, the conduit or the collection vessel.

A user, for example, a patient providing a breath sample or an individual connecting a collection vessel to a spectrometer, is provided a signal when a sought breath sample flow rate is flowing into the spectrometer. For example, the user is signaled that a target flow rate, a flow rate within a specified volume range, or a constant flow rate is achieved. Suitable signals include a visual output, for example, a needle, arrow, dial or a light indicating, for example, that the flow rate was held constant during sampling. Other suitable signals include noise or other output that signals the user that the sought flow rate is achieved. The measurement of a patients expired air may be clinically relevant, such as, for example, in the case of asthma patients. In this way, the patient's pulmonary function may be measured and the compounds in the patient's breath may be analyzed simultaneously.

For example, when a patient exhales a breath sample into a mouthpiece that provides a channel into a FAIM spectrometer, the patient is provided a signal regarding the flow rate of the patients exhaled breath. In one embodiment, when the patient exhales the breath sample, the patient is provided a visual signal of their breath flow rate as indicated by a needle of a flow meter visible to the patient providing the sample. The flow meter needle indicates to the patient that, for example, their sample is provided at a constant flow rate. This feedback or signal to the patient enables the patient to adjust their exhaling to provide the sought breath sample. The flow meter is placed in, for example, the channel the exhaled breath sample flows through into the spectrometer. Alternatively, the flow meter is disposed on, in or about the spectrometer housing. In embodiments where a collection vessel is employed, a flow meter is disposed in, on or about the collection vessel, spectrometer, the spectrometer housing, or the conduit connecting the collection vessel to the spectrometer. One or more flow meters may be simultaneously employed to detect the flow rate of a breath sample.

In another embodiment, only a fixed quantity, e.g., a slug, of breath sample is permitted to enter the spectrometer. For example, where the breath sample is provided from a collection vessel into the spectrometer, the one or more pumps that move the breath sample from the collection vessel into the FAIM spectrometer pump only a fixed quantity of breath sample into the spectrometer. Where the breath sample is provided from a patient into the spectrometer, for example, through the inlet of the spectrometer or via a channel attached to the spectrometer, a flow meter may be employed to permit only a fixed quantity of sample to enter the spectrometer.

In another embodiment, before the breath sample is introduced into the spectrometer, the breath sample is separated according to one or more methods of intermediate analytical separation. Suitable intermediate separation methods include classic analytical chemistry separation techniques. Such intermediate analytical techniques include gas chromatography, micro-gas chromatography, and mass spectroscopy. High Performance Liquid Chromatography (HPLC) provides another suitable intermediate separation method of, for example, condensed breath. Such pre-separation of chemicals in the sample enables more advanced analytical separation by providing improved FAIM spectrometry patterns that are distinct and distinguishable.

In one embodiment, the moisture and other compounds that are deemed to interfere with FAIM spectrometer analysis are filtered from the sample prior to sample introduction into the FAIM spectrometer. At least one of the collection vessel, the channel, or the conduit is adapted to filter the breath sample prior to introduction into the FAIM spectrometer. Suitable sample filters include, for example, moisture traps and solid phase microextraction equipment that selectively traps biomarkers from the sample.

Many factors contribute to natural variability among breath samples. For example, the proportion of alveolar air to dead-space air varies from breath sample to breath sample, which leads to highly variable quantitative data. To obtain a reproducible breath sample, standardized procedures for sample collection and treatment are implemented. The patient is instructed to breathe according to a standard protocol prior to providing the exhaled breath sample to be tested. According to one exemplary protocol, the patient rinses out the dead-space of the lungs by three or four breaths prior to sample collection. The sample is, for example, the latter half of an expired breath. Where breath samples collected without dead-space air enables monitoring CO₂ content of the sample or breath temperature.

Such breath sampling protocols may vary and may be dictated by the suspected medical condition, suspected type of chemical exposure, or the time since the exposure occurred. When exhaled breath is used to measure a process in the lung, exclusion of upper airway dead space gas is important. Such standard breathing protocols may not be necessary in all applications, for example, when the breath sample is analyzed for diseases in other organs.

It may be necessary to standardize the spectral normalization against the proportion of CO₂ that is present in a breath sample via mass spectrometry. Normalization of breath samples in this way is analogous to standardization of urinary compounds to creatinine concentration in urine analysis. Additionally, air samples from the surrounding environment are also collected and analyzed as blank controls to compare with the patient breath samples.

3. Breath Sample Analysis

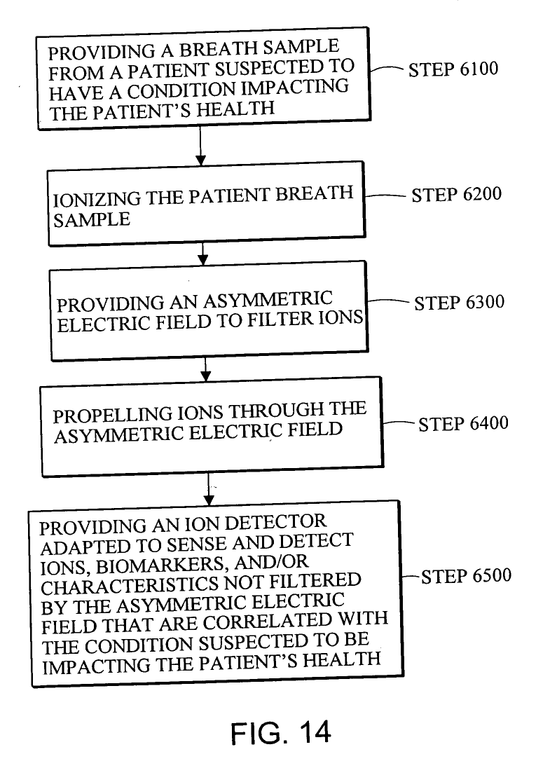

Patients suspected to have conditions impacting their health provide breath samples for detection, analysis and diagnosis by FAIM spectrometry. Generally, referring now to FIG. 14, a patient suspected to have a condition impacting their health provides a breath sample (STEP 6100). The FAIM spectrometer ionizes the patient breath sample (STEP 6200) and provides an asymmetric electric field to filter ions (STEP 6300). Ions propelled through the asymmetric electric field (STEP 6400) are sensed and detected by the ion detector. The provided ion detector is adapted to sense and detect ions, biomarkers, and/or characteristics not filtered by the asymmetric electric field that are correlated with the condition suspected to be impacting the patient's health (STEP 6500).

Specifically, an exhaled breath sample is taken from a patient suspected to have a pulmonary infection (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with pulmonary infection and pulmonary infectious disease that pass through and are not filtered by the asymmetric electric field (STEP 6500).

The classes of biomarkers indicative of pulmonary infection include, for example, bacterial infection, viral infection, fungal infection, yeast infection, infectious disease agents, pulmonary histoplasmosis, secondary infections associated with cystic fibrosis (CF), bronchiopulmonary aspergillosis, bronchiectasis, response to biowarfare agents, or emerging infectious disease agents. Emerging infectious disease agents include, for example, SARS. Biomarkers correlated with pulmonary disease and pulmonary infectious disease enable avoidance of confusion of a patient's condition with common respiratory ailments, such as, for example, pneumonia and influenza including Streptococcus pneumoniae, Staphylococcus aureus, Haemophilus influenzae and Mycoplasma pneumoniae. Biomarkers indicative of pulmonary infection include a host response, and/or general inflammation, in addition, elevated nitric oxide (NO), elevated carbon monoxide (CO) and volatile lipid peroxidation product levels may be sensed and measured by the FAIM spectrometer, providing a non-specific indicator of pulmonary inflammation. FAIM spectrometry is employed to analyze a patient's breath sample to identify pulmonary infection and the class of pulmonary infection in the sample. The FAIM spectrometer provides a non-invasive, rapid, and fieldable test. The FAIM spectrometer is applicable to mass casualty situations in ER settings. It provides the ability to distinguish between common pulmonary bacterial and viral infections from intentional pathogen infections, for example, inhalational anthrax.

For example, NO is measured as an indicator of pulmonary macrophage activation in pulmonary infection. Though not specific to a particular set of pulmonary infections, heightened concentrations of NO in the exhaled breath will serve as a signal of serious illness, cancer, cystic fibrosis, asthma, pulmonary inflammation and as an extremely sensitive indication of early pulmonary bacterial infection. The normal distribution of background spectral biomarkers is measured and determined in breath samples from healthy volunteers. The spectra from normal breath samples is spiked with clinically relevant increased levels of NO and compared with the spectra distribution from the healthy volunteers. The NO levels measured in samples from healthy volunteers is compared to the NO levels of emergency room patients with clinical, laboratory and radiographic findings consistent with bacterial pneumonia. The patient breath samples are obtained according to standardized protocol, described above.

In another embodiment, a patient suspected to have a metabolic disease provides a breath sample (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with metabolic disease (STEP 6500). Metabolic diseases that may be detected include diabetes, cerumloplasmin deficiency, phenylketonuria, Fabrys disease, Gauchers disease, alpha1 antitrypsin deficiency, bronchiolitis obliterans (BOOP), and maple syrup urine disease. The classes of biomarkers indicative of metabolic disease include, for example, ketones, acetone, and/or volatile organic compounds (VOC) from growth of bacteria in pulmonary space. FAIM spectrometry is employed to analyze a patient's breath sample to detect that the patient is suffering from a metabolic disease and to identify the disease.

An exhaled breath sample is taken from a patient suspected to have chronic progressive degenerative pulmonary disease (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with chronic progressive degenerative pulmonary disease (STEP 6500). Degenerative pulmonary diseases that may be detected include emphysema, chronic bronchitis, and Chronic Obstructive Pulmonary Disease (COPD). The classes of biomarkers indicative of chronic progressive degenerative pulmonary disease include, for example, heightened concentration of NO in exhaled breath consistent, carbon monoxide content, or hydrocarbons content consistent with COPD in exhaled breath. Elastin breakdown consistent with emphysema provides another biomarker indicative of chronic progressive degenerative pulmonary disease. FAIM spectrometry is employed to analyze a patient's breath sample to detect that the patient is suffering from a chronic progressive degenerative pulmonary disease and to identify the disease. In some cases, the breath sample may be condensed into a liquid phase and then revolatilized to concentrate biomarkers present in the lung.

In another embodiment, an exhaled breath sample is taken from a patient suspected to have lung cancer (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with lung cancer (STEP 6500). The classes of biomarkers indicative of lung cancer include, for example, methylethylketone, n-propanol, tolualdehyde, and oxepanone. FAIM spectrometry is employed to analyze a patient's breath sample and identify lung cancer in the sample.

In still another embodiment, an exhaled breath sample is taken from a patient suspected to suffer from organ dysfunction (step 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with organ dysfunction (STEP 6500). Organ dysfunctions that may be detected include, for example, acute or chronic disease onset, liver disease, heart attack, myocardial infarction, chronic cardiac disease, angina, kidney failure, bowel failure, pancreatic failure, endocrine dysfunction and certain mental disorders. The classes of biomarkers indicative of organ dysfunction include, for example, a rise in ketone level found in patient breath marks cardiac distress. Also, acute ischemia and a breakdown and peroxidation of cell lipids provide cardiac distress markers. The presence of pentane in breath is a marker for acute myocardial infarction. Ammonium provides a marker for kidney failure. FAIM spectrometry is employed to analyze a patient's breath sample to detect that the patient is suffering from organ dysfunction and to identify the organs that are dysfunctional. An increase in certain volatile biomarkers in schizophrenic patients is observable via gas chromatography-mass spectrometry (GC-MS).

In another embodiment, a patient receiving a course of drug therapy provides an exhaled breath sample (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with a response to drug therapy (STEP 6500). Classes of biomarkers indicative of the presence or absence of an ailment targeted for drug therapy treatment are evaluated. Alternatively, classes of biomarkers indicative of the presence of a drug therapy in the patient's body are evaluated. FAIM spectrometry is employed to analyze a patient's breath sample to identify and quantify markers in the sample indicative of the patient's response to drug therapy. In this way, the success of a targeted drug therapy may be evaluated by FAIM spectrometry.

In another embodiment, an exhaled breath sample is taken from a patient suspected to have been exposed to industrial chemicals (STEP 6100). The FAIM spectrometer ionizes the breath sample (STEP 6200), provides an asymmetric electric field to filter ions (STEP 6300), and propels ions through the asymmetric electric field (STEP 6400). The FAIM spectrometer contains an ion detector adapted to sense and detect ions and/or spectral biomarkers correlated with industrial chemicals (STEP 6500). A patient may carry, in the pulmonary space of his or her lungs, biomarkers indicative of chemical exposure that occurred hours, days, and weeks prior. For example, a patient may continue to exhale chemicals that she was exposed to one and even two or more days prior providing a breath sample. Classes of biomarkers that indicate a patient was exposed to industrial chemicals present in the environment include, for example, xylene, and toluene. FAIM spectrometry is employed to analyze a patient's breath sample to identify markers of industrial chemical exposure present in a patient breath sample. Optionally, the amount of chemical exposure may be quantified by FAIM spectrometry.

In another embodiment, FAIM spectrometry is employed for personnel identification. For example, a genetically determined unique signature may be sensed, detected, and identified by FAIM spectrometry. Such unique signature detection provides a method to identify individuals. In one embodiment, emanations (e.g., odors) from a human provide a distinctive identifying characteristic analogous to a unique fingerprint or signature. In one embodiment, major histocompatability complexes (MHC) related volatiles are analyzed, detected and employed to identify individuals. Such methods of personnel identification employing FAIM spectrometry to analyze body emanations may be employed in search and rescue missions in, for example, an urban setting. Another example of unique signature detection may be used in building air handling systems to monitor for building entry, for example, unlawful building entry.

FAIM spectrometry is employed to evaluate an exhaled breath sample for common respiratory ailments, such as, for example, pneumonia and influenza including Streptococcus pneumoniae, Staphylococcus aureus, Haemophilus influenzae and Mycoplasma pneumoniae.

FAIM spectrometers may be employed to analyze breath samples to detect, identify, and diagnose other conditions impacting patient health, such as, infectious disease, for example, tuberculosis. FAIM spectrometers may be used to analyze, identify, diagnose, or monitor conditions including bacterial colonization in the lungs of Cystic Fibrosis patients, pancreatic function in Cystic Fibrosis patients, carbon monoxide and/or hydrocarbons content in Cystic Fibrosis patients, carbon monoxide and/or hydrocarbons in asthma patients, carbon monoxide content in cancer patients, carbon monoxide content in bronchiectasis patients, anesthesia monitoring as part of, for example, ventillation system care, ketones and ketoacidosis indicative of starvation, bacterial irritable bowel syndrome markers, feculent amines in bowel obstructions, bacterial byproducts in anaerobic infections, bacterial overgrowth of the small bowel, exhaled hydrogen after a specific sugar or starch load to demonstrate lactose deficiency, ethane and pentane produced by the peroxidation of linoleic and linolenic acid, products of lipid peroxidation (e.g., pentane)polyunsaturated fatty acids found in cellular membranes that are oxidized during tissue ischemia and reperfusion injury, hydrocarbon content in exhaled breath and early pathogen infection response to bioweapons for example inhaled anthrax. FAIM spectrometers may also be employed to analyze traces of inhaled drugs (e.g., marijuana, cocaine, and heroine) in a patient sample. FAIM spectrometers may also be used to analyze the presence of pentane in patient sample, for example a breath sample, which provides a marker for arthritis and multiple sclerosis. The presence of ethane in a patient sample, such as a breath sample, indicates that a patient may be suffering from a vitamin E deficiency. The presence of heavy hydrocarbons are markers for prostate cancer and/or bladder cancer.

4. Biomarker Identification

Reference biomarkers and biomarker patterns are identified for use in correlating patient breath samples. In some cases patient breath samples are correlated with known reference biomarker patterns. Also, breath samples may be spiked with a known biomarker or know biomarkers and correlated to provide reference biomarker patterns. In one embodiment, metabolic bacterial reference biomarkers are identified by collecting gaseous headspace samples above vegetative liquid cell cultures. The headspace samples are correlated with biomarker patterns in patient breath samples.

For example, the reference biomarkers from early vegetative B. subtilis strain 168 (ATCC 23857), B. thuringiensis (ATCC 10792), and type III Streptococcus pneumoniae (ATCC 10813) in liquid culture may also be identified. The identified vegetative S. pneumoniae markers serve as controls for biomarkers from bacterial pneumonia patient breath samples. Bacterial cultures are maintained at log phase growth in 100 ml Erlenmeyer flasks optimized for anaerobic cultures and headspace sampling. Gaseous headspace samples are drawn off the liquid cultures in 4 hour intervals for the first 12 hours after culture inoculation, with a control measurement taken at time 0 hour. This sampling method identifies vegetative markers that may change with time as the bacteria proliferate. If there are radical differences in biomarkers present at time 0 hour and 4 hour, then the testing procedure is revised and samples are taken from the headspace gases every hour starting at inoculation. Similar techniques may be employed to identify reference biomarkers to be employed in accordance with the invention.

5. Design of Spectrometer

As discussed above, FAIM spectrometers may be micromachined. Such micromechanical FAIM spectrometers are portable. The FAIM spectrometer is, for example, hand held. In one embodiment, the portable FAIM spectrometer is powered by AC power supply. In another embodiment, the portable FAIM spectrometer is powered by an independent power supply. Suitable independent power supplies include battery power and power generators adapted to power the FAIM spectrometer.

The micromachined FAIM spectrometer may be adapted to be used in the field, thus enabling FAIM spectroscopy analysis to be provided in remote regions, in multiple regions, and in areas where such analysis is infrequent. Fieldable FAIM spectrometers can avoid the task of transporting samples from a sampling site into a laboratory for later analysis. The portable FAIM spectrometer is robust and sensitive enough for in field operation, analysis, detection, identification, quantification and diagnosis. In another embodiment, the portable FAIM spectrometer is designed to fit into a small space. For example, a fieldable FAIM spectrometer is adapted to fit into a bag or suitcase and optionally has an independent power supply, such as battery power. In one embodiment, the portable FAIM spectrometer fits into a backpack and is self-contained, featuring a battery power supply and a data collector, described below.

The FAIM spectrometer features a data collector. The data collector collects any ions sensed by the ion detector. The data collector may be, for example, separate from, integral with, or disposed on the FAIM spectrometer. In one embodiment, the FAIM spectrometer has a data port that links to a data collector to provide data regarding the ions sensed by the ion detector to the data collector. The data collector may be, for example, a personal data assistant or a computer such as a laptop computer. Suitable personal data assistants that may be employed in accordance with the invention are available from Palm® PDA (available from PALM Inc. Mountain View Calif.). Data collection may be employed to evaluate the data collected from the FAIM spectrometer for a pattern.

6. Data Analysis

Continued interrogation of breath samples from patients with a wide variety of ailments, for example a wide variety of pulmonary infections, enables the FAIM spectrometry device to learn from continued interrogation of patient samples. The spectrometer, data collector and data analysis systems are able to analyze, detect and identify a variety of health issues based on the measurement of pathogen specific markers. In certain embodiments, the spectrometer, data collector and data analysis systems are able to complete sampling, analysis, and identification at the point of care of a the patient being sampled.

Biomarker species contained in breath samples may be evaluated for one or more patterns e.g., fingerprinted. Multivariate data produced by the FAIM spectrometry device includes data parameters such as DC compensation voltage, RF field strength, and retention time. Such multivariate data is evaluated using bioinformatics tools. Suitable bioinformatics tools include genetic algorithms, adaptive pattern recognition, and cluster homogeneity analysis.

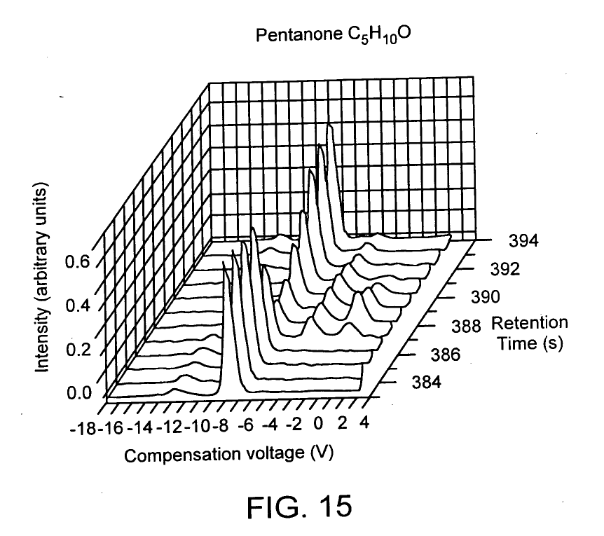

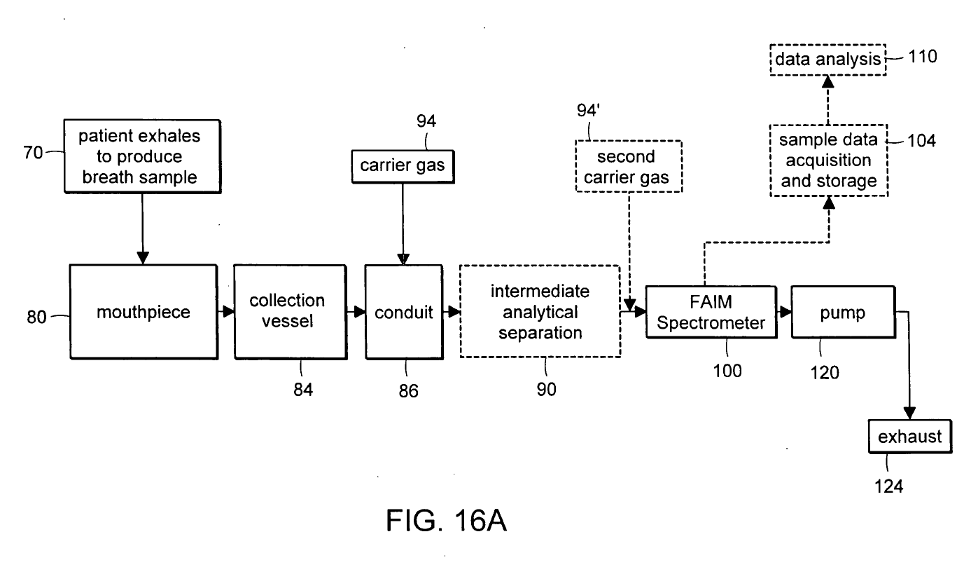

FIG. 15 provides one example of FAIM spectrometry data amenable to bioinformatics analysis. As shown in FIG. 15, pentanone, a biomarker of reperfusion injury, was measured at low concentrations using the FAIM spectrometry device. Rather than considering the pentanone measurement as a single data point represented by the compensation voltage resulting in maximum signal, the bioinformatics tool produces a pattern or fingerprint based on the three-dimensional set of spectra. FIG. 15 shows a plot of FAIM spectra data including as the y-axis, intensity (in arbitrary units), versus the x-axis, compensation voltage (in volts), as a function of the z-axis, retention time (in seconds), for pentanone. Such time series spectra may be employed for quantitative pattern recognition analysis. The use of retention time, field strength, and other variables available for analysis provides a rich data set amenable to powerful pattern recognition and cluster analysis tools.

Using “cluster analysis,” patterns of individual biomarkers are identified in a training group. For the purpose of these analyses, the decision boundary is defined as a 90% pattern match. If the data do not fall within the 90% decision boundary of any existing cluster in the model, it is used to establish a new cluster. The cluster map that best separates one group from the control is used for validation. Test samples not used during the training process may be analyzed, and the results from the testing set of data are used for determination of sensitivity, specificity and positive predictive value of the biomarker model. For example, cluster pattern analysis may be employed to identify specific biomarkers in breath exhalate samples for clinically confirmed viral influenza, viral pneumonia, and anaerobic bacterial infection patients.

Correlogic Systems, Inc. is a Maryland-based bioinformatics company that has developed pattern recognition and pattern discovery software with a wide variety of applications for bio-marker discovery, disease detection, and new drug discovery processes. Their software has been applied in the field of proteomics—the study of human protein data—with concentration on the early detection of prostate, ovarian, and other cancers. Correlogic's software makes it possible to identify proteomic bio-markers and other complex biomolecular relationships to help detect and cure life-threatening diseases. This technology can be used for pinpointing early indicators of various diseases. For analysis of complex biological data, the evolutionary component of the Correlogic's software first generates a set of candidate biomarkers. Each set of biomarkers is tested for its ability to distinguish diseased samples from healthy ones. The evolutionary algorithm iteratively processes a large number (15,000-20,000+) of the candidate biomarkers until it finds a set that optimally segments diseased from healthy samples. This software is fundamentally probabilistic—it works by randomly selecting candidate biomarkers and then repeatedly refining the population of selections. The software's evolutionary component improves the likelihood of identifying an optimal set of data points.

7. Exemplary Breath Analysis Systems

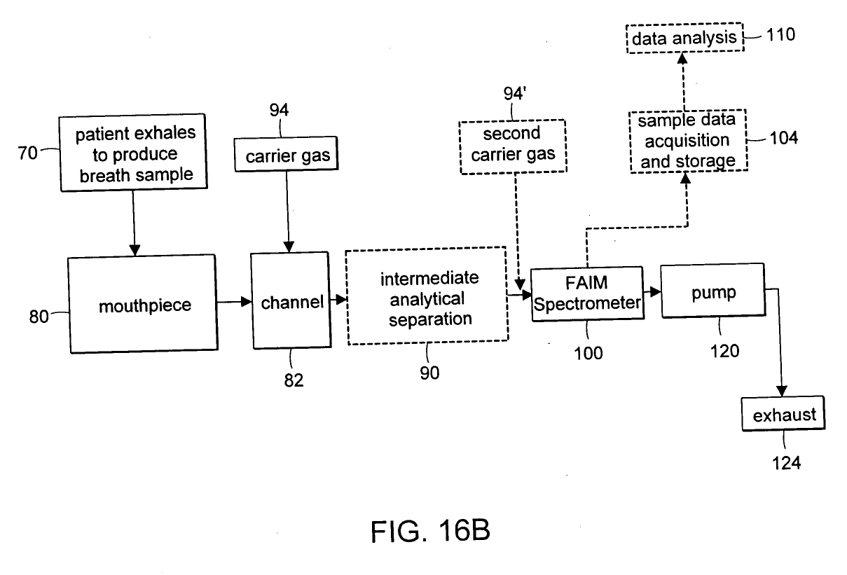

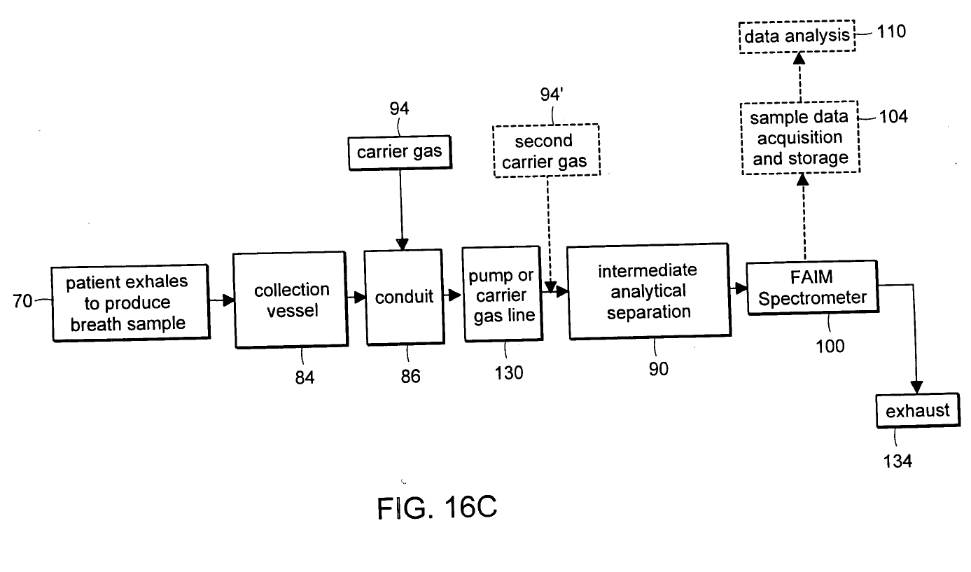

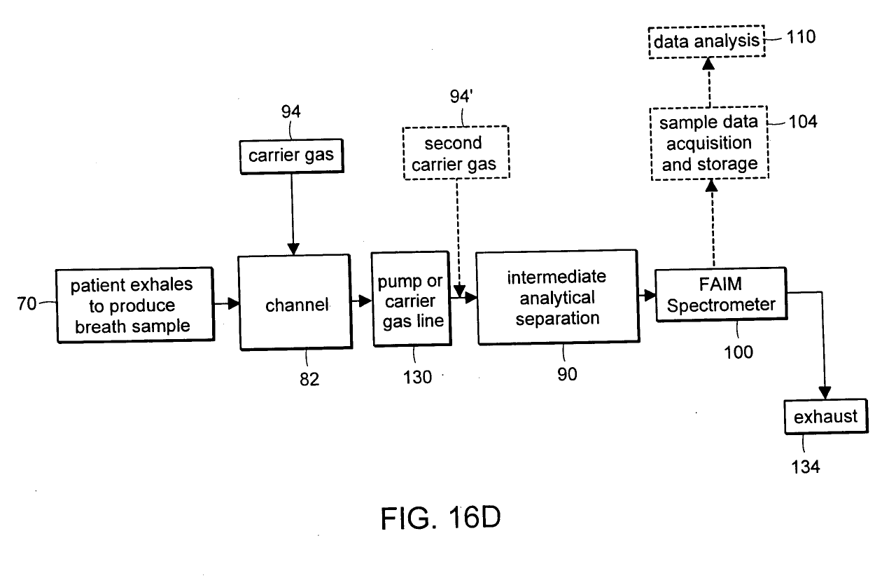

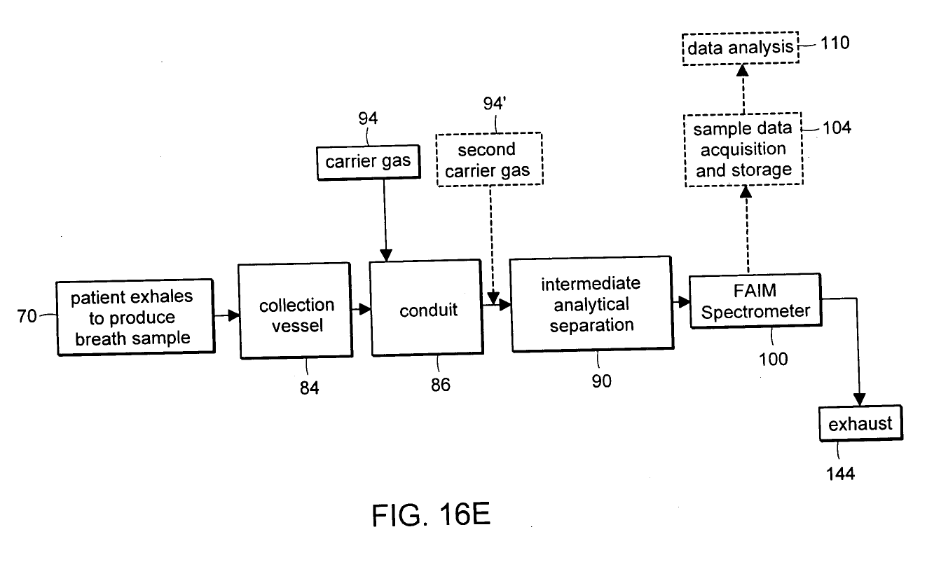

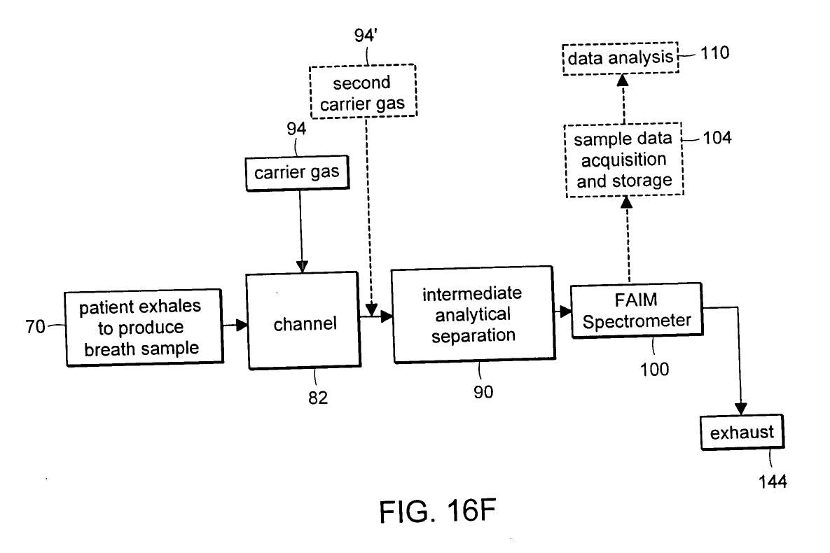

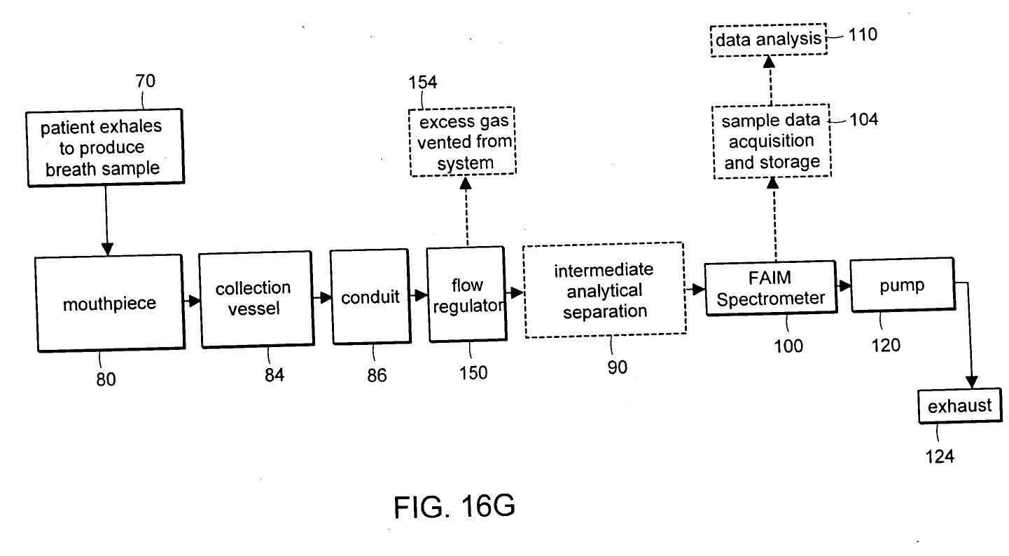

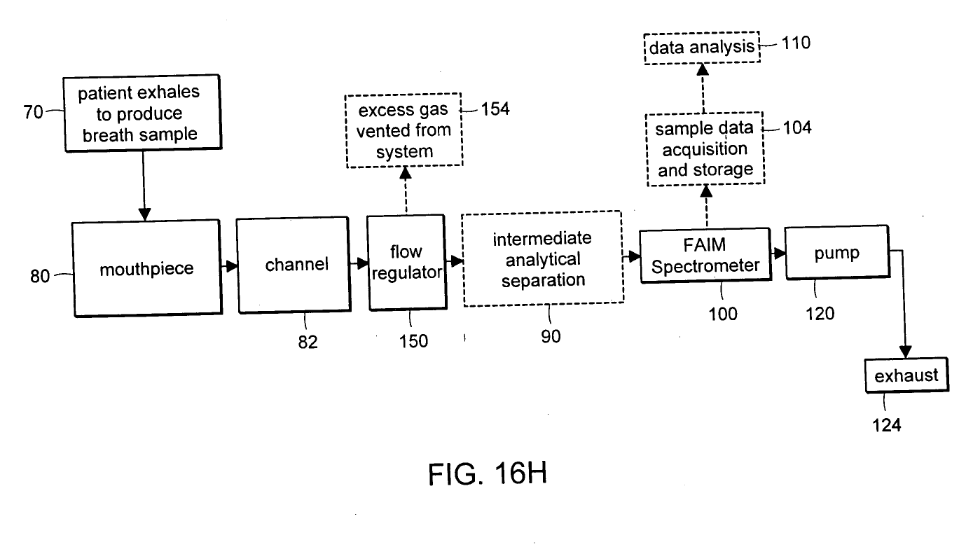

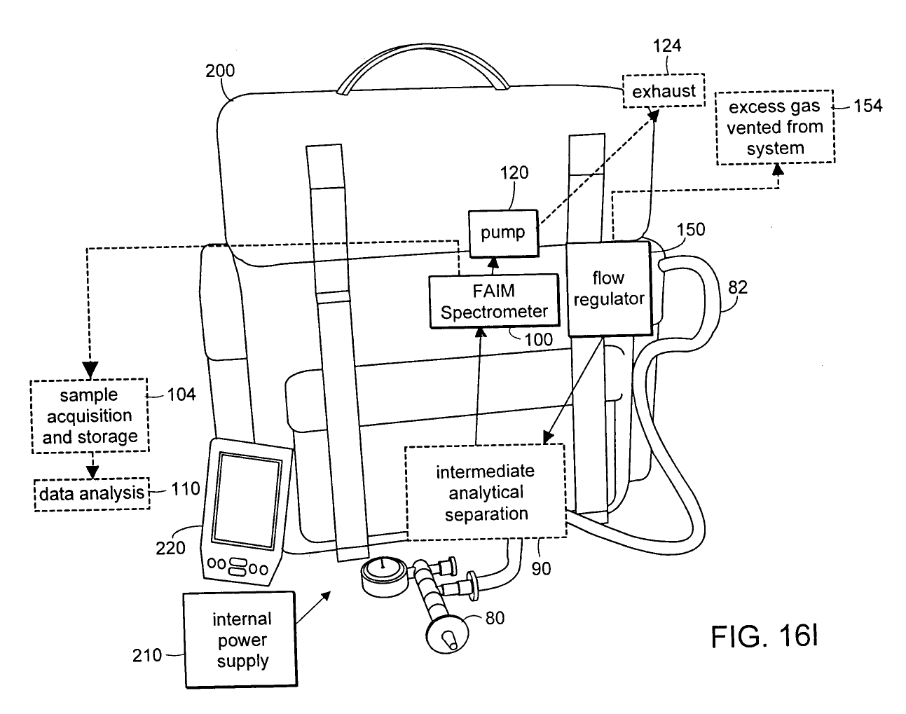

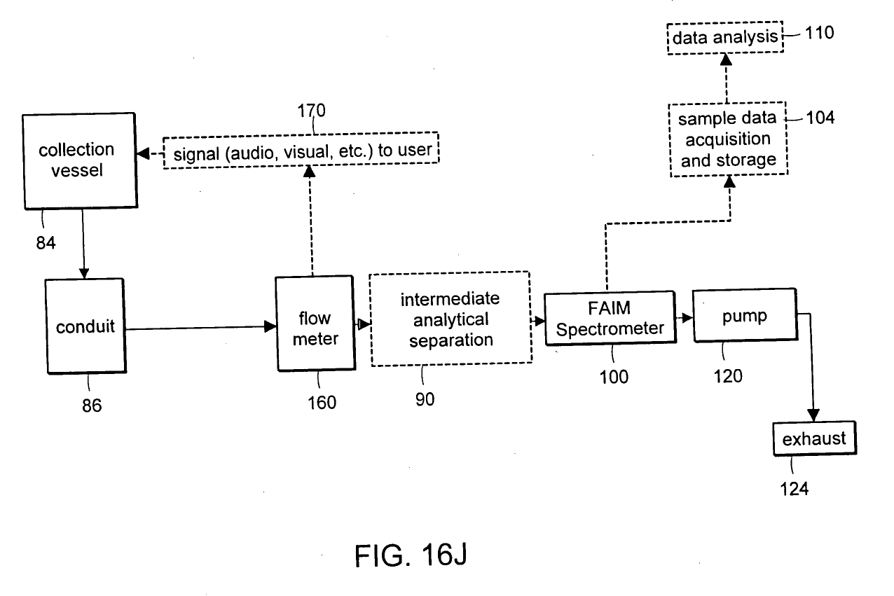

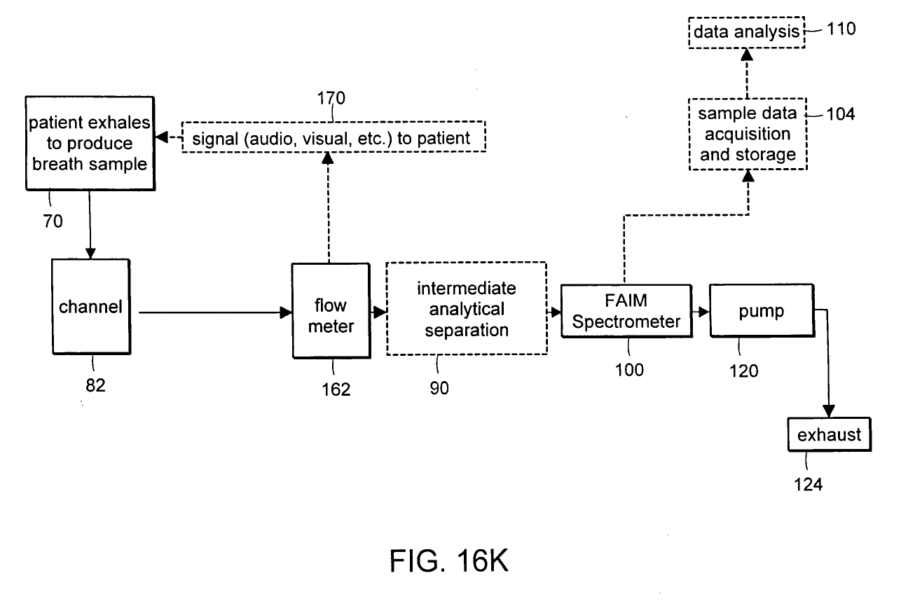

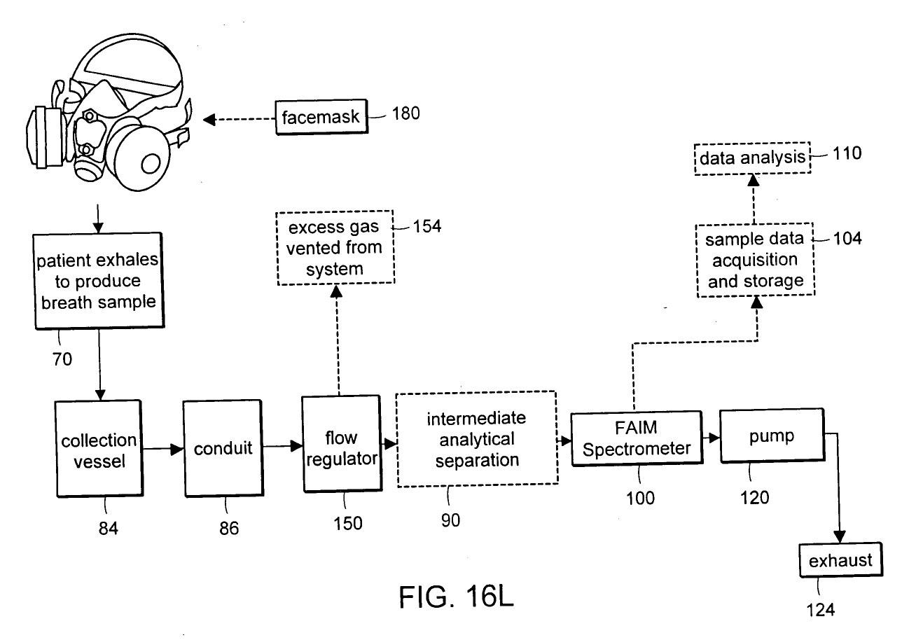

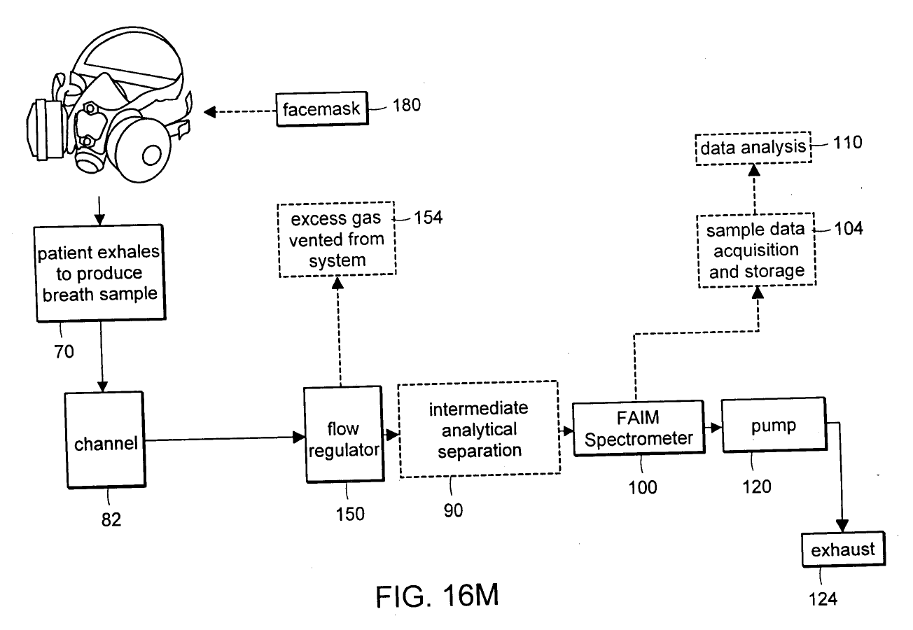

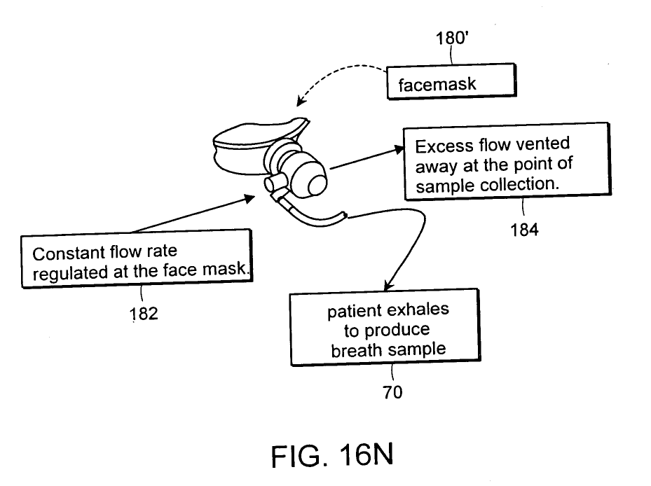

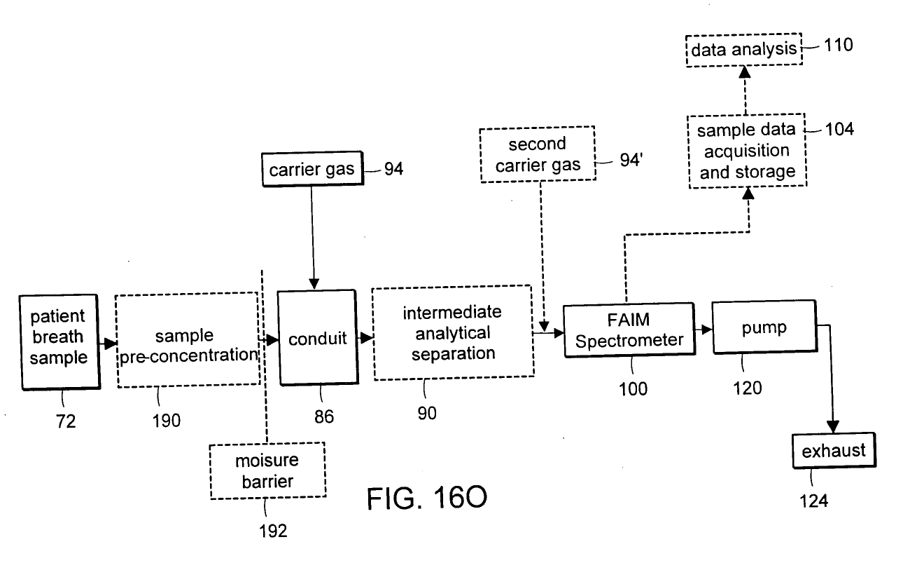

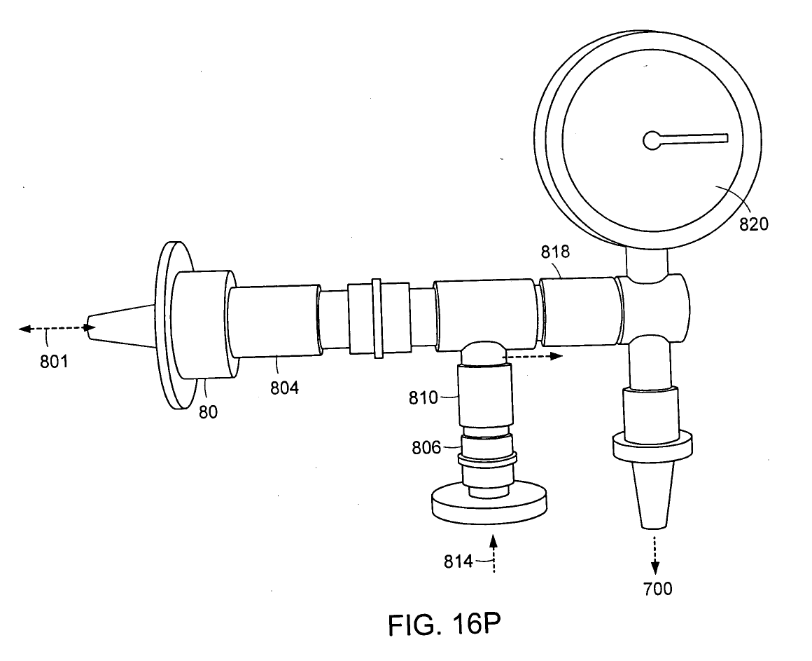

FIGS. 16A-16Q show breath analysis systems featuring various combinations of elements, described above, that, in combination, provide a system for breath analysis.

FIG. 16A shows a system for breath analysis in which a patient exhales to produce a breath sample 70 and the patient exhales into a mouthpiece 80. The mouthpiece 80 is disposed on, for example, the collection vessel 84 to collect the breath sample. A conduit 86 introduces the breath sample into the FAIM spectrometer 100. Optionally, as shown in phantom, the breath sample undergoes an intermediate analytical separation 90 by, for example, a suitable classical analytical chemistry technique prior to entering the FAIM spectrometer. A carrier gas 94 enters the system through conduit 86. Optionally, as shown in phantom, a second point of entry of carrier gas 94′ is just prior to entry of the breath sample into the FAIM spectrometer 100. In one embodiment, the carrier gas 94 and the second carrier gas 94′ are the same gas. In another embodiment, the carrier gas 94 is different from the second carrier gas 94′, A pump 120 draws the sample and the carrier gas 94 and 94′ through the FAIM spectrometer 100 at, for example, a constant flow rate. In one embodiment, the pump 120 is external and positioned after the FAIM spectrometer. Exhaust 124 exits from the system from, for example, the pump 120. Optionally, as shown in phantom, the breath sample data is acquired and stored 104 thereafter the data is analyzed 110. The data from the FAIM spectrometer 100 may be analyzed in, for example, “real time” (e.g., concurrent with the patient exhaling the breath sample 70).

FIG. 16B shows a system for breath analysis that is substantially similar to the system of FIG. 16A, but has a few differences. When the patient exhales to produce a breath sample 70, the patient exhales into a mouthpiece 80 that is disposed on, connected to, or forms a channel 82 that introduces the breath sample into the FAIM spectrometer 100. A carrier gas 94 enters the system through channel 82. Optionally, as shown in phantom, the breath sample undergoes an intermediate analytical separation 90. Also, as shown in phantom, an optional second point of entry of carrier gas 94′ is just prior to entry of the breath sample into the FAIM spectrometer 100. A pump 120, for example an external pump, draws the sample and the carrier gas 94 and 94′ through the FAIM spectrometer 100. Exhaust 124 exits from the system from, for example, the pump 120. The breath sample data is optionally acquired (from the FAIM spectrometer 100) and stored 104, thereafter the data is analyzed 110 in, for example, “real time.”

FIG. 16C shows a system for breath analysis in which a patient exhales a breath sample 70 into a collection vessel 84. A conduit 86 introduces the breath sample from the collection vessel 84 into a pump or carrier gas line 130. A carrier gas 94 enters the system through conduit 86. Thereafter, the breath sample travels into an intermediate analytical separator 90. Optionally, as shown in phantom, a second point of entry of carrier gas 94′ is just prior to entry of the breath sample into the intermediate analytical separator 90. After the breath sample has undergone intermediate analytical separation 90 the breath sample enters the FAIM spectrometer 100. Exhaust 134 exits from the system from the FAIM spectrometer 100. The pump 130 pushes the breath sample and carrier gases through the intermediate analytical separation 90 and through the FAIM spectrometer 100. The breath sample data is optionally acquired (from the FAIM spectrometer 100) and stored 104, thereafter the data is analyzed 110.

FIG. 16D shows a system for breath analysis that is substantially similar to the system of FIG. 16C, but has a few differences. When the patient exhales to produce a breath sample 70, the patient exhales into a channel 82 that is disposed or connected to and introduces the breath sample into the pump or carrier gas line 130. A carrier gas 94 enters the system through channel 82. Thereafter, the breath sample travels into an intermediate analytical separator 90. Optionally, as shown in phantom, a second point of entry of carrier gas 94′ is just prior to entry of the breath sample into the intermediate analytical separator 90. After the breath sample has undergone intermediate analytical separation 90 the breath sample enters the FAIM spectrometer 100. Exhaust 134 exits from the system from the FAIM spectrometer 100. The pump 130 pushes the breath sample and carrier gases through the intermediate analytical separation 90 and through the FAIM spectrometer 100. The breath sample data is optionally acquired (from the FAIM spectrometer 100) and stored 104, thereafter the data is analyzed 110.

FIG. 16E shows a system for breath analysis in which a patient exhales a breath sample 70 into a collection vessel 84 to collect the breath sample. A conduit 86 introduces the breath sample into the intermediate analytical separation 90. A carrier gas 94 enters the system through conduit 86. Optionally, as shown in phantom, a second point of entry of carrier gas 94′ is just prior to entry of the breath sample into the intermediate analytical separation 90. Pressure from the carrier gas 94 and optionally the carrier gas 94′ pushes the breath sample through the intermediate analytical separation 90 and into the FAIM spectrometer 100. Exhaust 144 exits the system via, for example, an outlet on the FAIM spectrometer 100. Optionally, as shown in phantom, the breath sample data is acquired (from the FAIM spectrometer 100) and stored 104, thereafter the data is analyzed 110.

FIG. 16F shows a system for breath analysis that is substantially similar to the system of FIG. 16E, but has a few differences. When the patient exhales to produce a breath sample 70, the patient exhales into a channel 82 that introduces the breath sample into the intermediate analytical separation 90. A carrier gas 94 enters the system through channel 82. Also, as shown in phantom, an optional second point of entry of carrier gas 94′ is just prior to entry of the breath sample into the intermediate analytical separation 90. Pressure from the carrier gas 94 and optionally the carrier gas 94′ pushes the breath sample through the intermediate analytical separation and into the FAIM spectrometer 100. Exhaust 144 exits the system from, for example, the FAIM spectrometer 100. The breath sample data is optionally acquired (from the FAIM spectrometer 100) and stored 104, thereafter the data is analyzed 110.

FIG. 16G shows a system for breath analysis in which a patient exhales a breath sample 70 into a mouthpiece 80. The mouthpiece 80 is disposed on, for example, the collection vessel 84 to collect the breath sample. A conduit 86 introduces the breath sample from the collection vessel 84 into a flow regulator 150. Here the patient's exhaled breath pushes the breath sample through the system pathway without any additional carrier gasses. The flow regulator 150 actively ensures that there is a constant flow through the remaining portions of the breath analysis system. As shown in phantom, any excess gas 154 is optionally vented from the system via flow regulator 150. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separation. Exhaust 124 exits the system from, for example, the pump 120. The breath sample data is optionally acquired (from the FAIM spectrometer 100) and stored 104, thereafter the data is analyzed 110.

FIG. 16H shows a system for breath analysis that is also substantially similar to the system of FIG. 16G, but has a few differences. When the patient exhales to produce a breath sample 70, the patient exhales into a mouthpiece 80 that is disposed on, connected to, or forms a channel 82 that introduces the breath sample into the flow regulator 150. The patient's exhaled breath pushes the breath sample through the system pathway without any additional carrier gasses. The flow regulator 150 actively ensures that there is a constant flow through the remaining portions of the breath analysis system. As shown in phantom, any excess gas 154 is optionally vented from the system via flow regulator 150. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separation 90. Exhaust 124 exits the system from, for example, the pump 120. The breath sample data is optionally acquired (from the FAIM spectrometer 100) and stored 104, thereafter the data is analyzed 110.

FIG. 16I shows a system for breath analysis that is substantially similar to the system of FIG. 16H and is portable (e.g., fieldable). A portable bag 200, for example a back pack, features the elements of the system of FIG. 16H and also features an internal power supply 210 and a Personal Data Assistant (PDA) 220. Some of the elements of the system are placed exterior to the bag 200 and others are carried inside the bag 200. Certain of the elements of the system are inside or exterior to the bag depending on user preference. In the embodiment shown in FIG. 16I, the mouthpiece 70 is exterior to the bag 200. When the patient exhales to produce a breath sample, the patient exhales into a mouthpiece 80. The mouthpiece is disposed on the channel 82. At least a portion of the channel is exterior to the bag 200. The channel introduces the breath sample into the flow regulator 150, which is housed inside the bag 200. The patient's exhaled breath pushes the breath sample through the system pathway without any additional carrier gasses. The flow regulator 150 actively ensures that there is a constant flow through the remaining portions of the breath analysis system. As shown in phantom, any excess gas 154 is optionally vented from the system via flow regulator 150. The bag 200 may contain one or more holes by which the excess gas vents from the system. Optionally, the breath sample travels into an intermediate analytical separator 90, which is also housed inside the bag. The breath sample then enters the FAIM spectrometer 100, also housed inside the bag. A pump 120, which may be inside the bag 200 but external to the FAIM spectrometer, may be employed to draw the sample through the FAIM spectrometer 100 and optionally through the intermediate analytical separation 90. Exhaust 124 exits the system from, for example, the pump 120 by one or more holes present in the bag. The breath sample data is optionally acquired (from the FAIM spectrometer 100) and stored 104, thereafter the data is analyzed 110. The data may be stored in and analyzed by the PDA 220, which may be inside or external to the bag 200. An internal power supply 210 powers the system by an independent power source, for example a battery.

FIG. 16J shows a system for breath analysis in which a conduit 86 introduces a breath sample from the collection vessel 84 into a flow meter 160. Here the patient's exhaled breath pushes the breath sample through the system pathway without any additional carrier gasses. The flow meter 160 measures the flow rate of the sample. The flow meter 160 optionally produces a signal 170, such as, for example, a noise or visual signal that indicates the flow rate to the user. The user, who is transferring the breath sample from the collection vessel 84 through the conduit 86 and into the remaining parts of the system (e.g., the intermediate analytical separator 90 and the FAIM spectrometer 100), is signaled 170 regarding the flow rate of the breath sample being transferred. For example, the user is signaled that the breath sample is flowing at a constant rate. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separator 90. Exhaust 124 exits the system from, for example, the pump 120. The breath sample data is optionally acquired (from the FAIM spectrometer 100) and stored 104, thereafter the data is analyzed 110.

FIG. 16K shows a system for breath analysis that is substantially similar to the system of FIG. 16J, but has a few differences. The patient exhales a breath sample 70 into a channel 82 that introduces the breath sample into the flow meter 162. The patient's exhaled breath pushes the breath sample through the system pathway without any additional carrier gasses. The flow meter 162 measures the flow rate of the sample. The flow meter 162 optionally produces a signal 170, such as, for example, a noise or visual signal that indicates the flow rate to the patient. The patient, who is exhaling the breath sample through the conduit 82 and into the remaining parts of the system (e.g., the intermediate analytical separator 90 and the FAIM spectrometer 100), is signaled 170 regarding the flow rate of their breath sample. For example, the patient is signaled that their breath sample is flowing at a constant rate. The patient can adjust their exhalation is response to the provided signal 170. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separator 90. Exhaust 124 exits the system from, for example, the pump 120. The breath sample data is optionally acquired (from the FAIM spectrometer 100) and stored 104, thereafter the data is analyzed 110.

FIG. 16L shows a system for breath analysis in which a patient wearing a facemask 180 exhales a breath sample 70 into a collection vessel 84. The facemask 170 filters incoming atmospheric air from, for example, contaminant. The facemask also monitors the patient's breath for patient health status. The collection vessel 84 collects the breath sample and a conduit 86 introduces the breath sample from the collection vessel 84 into a flow regulator 150. Here the patient's exhaled breath pushes the breath sample through the system pathway without any additional carrier gasses. The flow regulator 150 actively ensures that there is a constant flow through the remaining portions (e.g., the optional intermediate analytical separation 90 and the FAIM spectrometer 100) of the breath analysis system. As shown in phantom, any excess gas 154 is optionally vented from the system via flow regulator 150. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separation 90. Exhaust 124 exits the system from, for example, the pump 120. The breath sample data is optionally acquired (from the FAIM spectrometer 100) and stored 104, thereafter the data is analyzed 110.

FIG. 16M shows a system for breath analysis that is substantially similar to the system of FIG. 16L, but has a few differences. The patient wearing a facemask 180 exhales a breath sample 70 into a channel 82. The facemask 170 filters incoming atmospheric air from, for example, contaminant. The facemask also monitors the patient's breath for patient health status. The channel 82 introduces the breath sample into the flow regulator 150. The patient's exhaled breath pushes the breath sample through the system pathway without any additional carrier gasses. The flow regulator 150 actively ensures that there is a constant flow through the remaining portions of the breath analysis system. As shown in phantom, any excess gas 154 is optionally vented from the system via flow regulator 150. Optionally, the breath sample travels into an intermediate analytical separator 90. The breath sample then enters the FAIM spectrometer 100. A pump 120, for example an external pump, may be employed to draw the sample through the FAIM spectrometer 100 and optionally through the intermediate analytical separation 90. Exhaust 124 exits the system from, for example, the pump 120. The breath sample data is optionally acquired (from the FAIM spectrometer 100) and stored 104, thereafter the data is analyzed 110.

FIG. 16N shows an alternate facemask 180′. The facemask 180′ may be employed, for example, in place of the facemask 180 described above with reference to FIGS. 16L and 16M. Facemask 180′ is a base face mask collection system that is adapted provide a patient breath sample 70. Facemask 180′ regulates breath sample flow rate 182 and is capable of venting excess flow away from the facemask 180′ so that such excess breath flow is not placed at that point of sample collection 184. The facemask 180′ features flow rate regulation 182 that is adapted to keep flow rate at, for example, a constant rate, a target rate, or within a rate range.

FIG. 16O shows a system for breath analysis in which a patient breath sample is provided 72. The patient breath sample may be directly from the patient or it may be from a collection vessel. As shown in phantom, the breath sample optionally undergoes sample pre-concentration 190. The pre-concentrator 190 may be used, for example, to concentrate low abundance compounds in the breath sample, thereby improving analysis. Thereafter, the breath sample optionally travels through a moisture barrier 192, as shown in phantom. The moisture barrier 192 may be employed to refine the sample prior to analysis, thereby improving analysis. Such sample pre-concentrators 190 and moisture barriers 192 may be employed as elements of any of the systems for breath analysis. After travelling through the moisture barrier 190 the breath sample travels through a conduit 86 or alternatively through a channel (not shown), which introduces the breath sample into the FAIM spectrometer 100. Optionally, as shown in phantom, the breath sample undergoes an intermediate analytical separation 90 by, for example, a suitable classical analytical chemistry technique prior to entering the FAIM spectrometer 100. A carrier gas 94 enters the system through conduit 86. Optionally, as shown in phantom, a second point of entry of carrier gas 94′ is just prior to entry of the breath sample into the FAIM spectrometer 100. In one embodiment, the carrier gas 94 and the second carrier gas 94′ are the same gas. In another embodiment, the carrier gas 94 is different from the second carrier gas 94′, A pump 120 draws the sample and the carrier gas 94 and 94′ through the FAIM spectrometer 100 at, for example, a constant flow rate. In one embodiment, the pump 120 is external and positioned after the FAIM spectrometer. Exhaust 124 exits from the system from, for example, the pump 120. Optionally, as shown in phantom, the breath sample data is acquired and stored 104 thereafter the data is analyzed 110. The data from the FAIM spectrometer 100 may be analyzed in “real time”.

FIG. 16P shows an embodiment of breath analysis collection interface. The collection interface includes a mouthpiece 80, which may be disposable. The patient can both breath through and exhale out or into the mouthpiece 80 through the mouthpiece inlet 801. After the breath sample flows into the mouthpiece inlet 801 it travels through an antibacterial filter 804. Air travels into the breath analysis collection interface through the incoming air inlet 814, Air travels through antibacterial filter 806 and through a unidirectional valve 810 to meet the breath sample traveling through the breath analysis collection interface, perpendicular to the incoming air inlet 814. The breath sample and the incoming air meet and travel thorough unidirectional valve 818. The breath sample exits the collection interface via outlet 700. The flow meter 820 evaluates the flow rate of the breath sample as the breath sample travels between unidirectional valve 818 and outlet 700. Such a breath analysis collection interface may be employed as an element of any of the systems for breath analysis. The breath sample that exits the outlet 700 may enter, for example, a collection vessel, a conduit, a channel, a intermediate analytical separation or a FAIM spectrometer.

FIG. 16Q shows an embodiment of a system for breath analysis including a facemask 180″ linked to a ventilator 188. As shown, the patient exhales a breath sample into the facemask 180″ and the breath sample flows through a channel 82. The breath sample enters the FAIM spectrometer 100 through the channel 82. The ventilator 188 is adapted to connect to the channel 82 and to introduce the breath sample to the FAIM spectrometer 100. The ventilator 188 sample is analyzed to track patients that are on a ventilator for the addition or disappearance of relevant biomarkers. For example, a patient in a critical care unit may be monitored for an aspiragillus infection after an organ transplant or after chemotherapy. Also, a patient's exhaled rate of anesthesia may be monitored during and after a surgical procedure.

8. FAIM Spectrometer Analysis and Clinical Data

After FAIM spectrometer analysis, retrospective clinical data is obtained (blood tests, culture results, etc). Clinical diagnoses, particularly those supported by specific laboratory diagnosis, are used to segregate patients into various groups. (i.e. pneumococcal pneumonia supported by culture results; influenza pneumonia or upper respiratory infection, supported by testing for Influenza virus). FAIM spectrometry patterns are analyzed using “cluster analysis” for specific diagnostic groups (pneumococcal pneumonia and influenza).

9. Environmental Sample Analysis

A FAIM spectrometer may be adapted to test the environment for, for example, pathogens and/or spores. A single FAIM spectrometer may be adapted to test both the environment and a patient sample, for example, a patient breath sample. In one embodiment, a FAIM spectrometer analyzes a breath sample taken from a patient suspected to have been exposed to industrial chemicals and the spectrometer also analyzes a sample from the environment, for example, an air sample, where the suspected exposure occurred.

Aerosolized endospore samples have distinctive biological markers that may be analyzed by a FAIM spectrometer, such aerosolized pathogens include Bacillus anthracis (e.g., B. anthracis or inhalational anthrax).

The capability of B. anthracis to form highly resistant spores makes it a prime candidate for an easily released biological weapon and it is among the category A pathogens listed by the Centers for Disease Control and Prevention. Early detection of such pathogens permits quick characterization of a threat versus a hoax, minimizing human casualties and reducing the time and financial burdens associated with containment, triage and clean up. Portable point-of-care technology to diagnose patient infection enables time sensitive therapeutic measures to be taken. Early and accurate detection avoids unnecessary treatment and reduces unnecessary strain on emergency room and other health care facilities. Currently, there is no multipurpose analyzer or microanalyzer capable of specific and sensitive environmental pathogen detection and early diagnosis of host response to infection.

Over the past two decades, scientists have adapted molecular biology techniques to detect B. anthracis using DNA-based, antibody-based and mass spectrometry analysis approaches. These tests vary greatly in sensitivity, response time, cost, availability and complexity of use. With the identification of species-specific primers, rapid polymerase chain reaction (PCR) has identified specific Bacillus species from both environmental and clinical samples. One novel detection method uses DNA-aptamers conjugated to magnetic electro-chemiluminescent beads to bind and detect Sterne strain B. anthracis spores.

Minisequencing on microchips containing gel-immobilized oligonucleotides has identified B. anthracis by single-nucleotide polymorphism (SNP) analysis, and several commercial PCR kits/plafforms are available that differ in sensitivity depending on sample type and preparation. The “Mayo-Roche Rapid Anthrax Test” is based on rapid-cycle real-time PCR and was developed as a collaborative effort. Although the “Mayo-Roche Rapid Anthrax Test” platform will yield results in approximately 35 minutes, anecdotal evidence suggests variable field results.

Antibody-based methods traditionally use fluorescent-conjugated antibodies to spore coat proteins to detect low levels of Bacillus spores. Phillips and Martin (1983) showed that it is possible to detect Bacillus spores with specificity using fluorescence in-conjugated polyclonal antibodies directed towards the spore coat. However, they also found that multiple anthrax serotypes exist among B. anthracis strains, making specific detection with this method difficult. More recently, monoclonal and polyclonal antibodies have been produced against Bacillus epitopes. These distinguish moderately well between B. anthracis and B. subtilis, but less effectively between B. anthracis and B. cereus spores. Additionally, variability still exists in the specificity of antibodies between spore coat and vegetative cell epitopes. Nevertheless, several novel antibody-based assays have been developed to identify Bacillus species. The electrochemiluminescent immunoassay (ECLIA) is based on a redox reaction between ruthenium (II)-trisbipyridyl Ru[(bpy)₃]²⁺ labeled antibody and the excess of tripropylamine, which generates photons. The magnetic particle fluorogenic immunoassay (MPFIA) technique employs antibody-coated magnetic beads as solid phase in suspension for bacterial capture and concentration in a 96-well microplate format¹⁶. Both the ECLIA and MPFIA are fast, but still require almost double the time of rapid PCR-based tests. Antibodies have also been immobilized onto solid substrates such as silicon chips or membranes for higher-throughput screening of environmental samples. A major limitation of these methods involves the specificity of the antibodies selected for use. However, fluorescent-labeled phage antibodies have recently been produced, and show promise as Bacillus species-specific markers.

Several chemical and analytical detectors are presently being investigated to rapidly identify Bacillus spores. Virtually all gas chromatograph (GC) detectors, such as the widely used flame ionization detector (FID), produce a signal indicating the presence of a compound eluted from the column. However, GC results lack the specific information required for unambiguous compound identification. An expedient and simple method for identification of unknown analytes requires a detector to provide an orthogonal set of information for each chromatographic peak. The mass spectrometer (MS) is generally considered one of the most definitive detectors for compound identification, as mass spectra generates a fingerprint pattern of fragment ions for each GC elutant. MS information is often sufficient for sample identification through comparison to compound libraries, and has been used to identify species of bacteria. Bacterial cell extracts themselves have been shown to produce reproducible spectra comprised mainly of phospholipids, glycolipids, and proteins. As such, this is a very sensitive method for identifying Bacillus species, and unique biomarkers have even been identified between closely related B. cereus strains. The so-called “tandem” MS method has yielded a wealth of specifically identified protein biomarkers for B. cereus using bioinformatic approaches. The matrix-assisted laser desorption/ionization mass spectrometry (MALDI-MS) has also shown that very low mass biomarkers between 2-4 kDa distinguish B. anthracis from other closely related Bacillus species. While this result was obtained using a very specific carrier matrix, it demonstrates that species-specific markers can exist if sample preparation is optimized. However, minor variations in sample/matrix preparations for MALDI-MS can produce significant changes in observed spectra. Finally, MALDI-MS has been shown to distinguish bacteria in aerosolized samples, albeit from radically divergent species, in a continuous fashion. Mass spectrometers are expensive, costing on average between about $50,000 and about $75,000 and mass spectrometer size remains relatively large, typically benchtop sized, making them difficult to deploy in the field. Mass spectrometers need to operate at low pressures and their spectra can be difficult to interpret. As discussed above, with FAIM spectrometers even larger numbers of species may be distinguished, as the spectra is more easily deconvoluted than those of MS due to differing ion mobilities.

A classic signature of bacterial spores from species such as Bacillus and Clostridium is the presence of high concentrations of 2,6-pyridinedicarboxylic acid, or dipicolinic acid (DPA). Typical spores contain roughly 5-15% dry weight of DPA (MW=167), which is speculated to provide the spore with heat resistance. Preliminary research has shown that pyrolysis of B. subtilis spores produces large quantities of gaseous DPA, which may then be detected by a standard gas chromatography-mass spectrometry (GC-MS) or by the FAIM spectrometer device. While the presence of DPA does not signify with certainty that a bioweapons agent such as anthrax is present in the environment, a sudden increase in DPA concentration can serve as a trigger for initiation of a target-specific detection cycle.

Solid spore samples may be analyzed by FAIM spectrometry via two potential sample introduction methods. First, pyrolysis is used to convert the spore sample into its component substances through the use of heat. The process generally leads to breakdown of the sample into smaller molecular components, giving rise to profiles that are characteristic of the sample in either its products or their relative distribution. Pyrolysis can be employed for identification and quantization of samples through the analysis of both the parent and the product ions. Detection of bacterial spore biomarkers by pyrolysis has been demonstrated using Gas Chromatography-Ion Mobility Spectrometry and Mass Spectrometry. Pyrolysis is a viable sample handling method for FAIM spectrometer analysis.

Characteristic bacterial biomarkers, in both the spore and vegetative state, are identified using FAIM spectrometry. Solid Bacillus endospore samples contain a multitude of species-specific biomarkers that can be tested by FAIM spectrometry. Specific spore sample handling and preparation techniques are employed to yield these markers.

A pyrolysis-FAIM spectrometry experimental setup produces spectral data from solid spore samples. A complete Pyrolysis-FAIM spectrometer system was assembled by coupling a commercial pyrolyzer with the necessary functions to handle the introduction of liquid and solid samples into the FAIM spectrometer by a pyrolysis-FAIM spectrometer interface. A pyrolysis protocol was obtained from the manufacturer of the pyrolysis system, available from CDS Analytical. The pyrolysis protocol was evaluated and optimized using Ion Trap Mass Spectrometry. The protocols were tested with dipicolinic acid (DPA), picolinic acid (PA) and pyrolyzed Bacillus subtilis samples (as a simulant for B. anthracis). The pyrolyzer is capable of heating samples from room temperature to 1400° C. at rates from 1 to 20° C./s. The controlled temperature ramping enables selective desorption of compounds from the probe, therefore enhancing resolution and signal-to-noise of the FAIM spectrometer. A drying function evaporates and vents the solvent out a purge vent resulting in sample concentration and prevention of the solvent from entering the FAIM spectrometer filter. A probe cleaning function flash-heats and desorbs residual sample between analyses. The pyrolate is transferred to the FAIM spectrometer through the pyrolysis-FAIM spectrometer interface, a sealed and heated interface. During sample loading on the probe, the pyrolysis chamber is purged while a stream of N₂ is diverted into the FAIM spectrometer. During pyrolysis, the flows are diverted through a 6-port valve into the FAIM spectrometer for introduction of the pyrolate into the FAIM spectrometer.

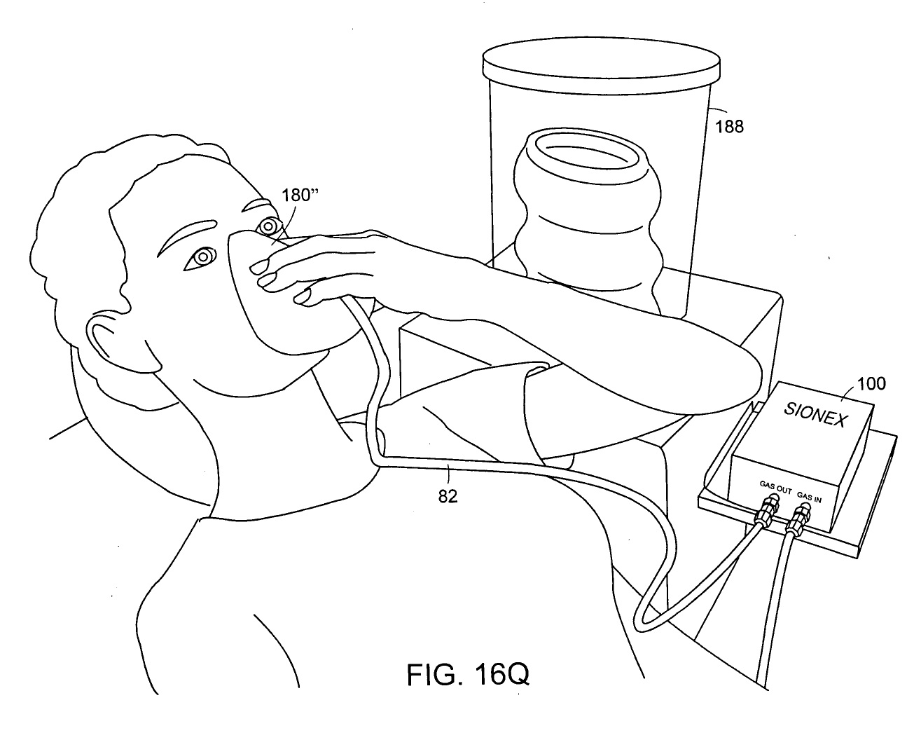

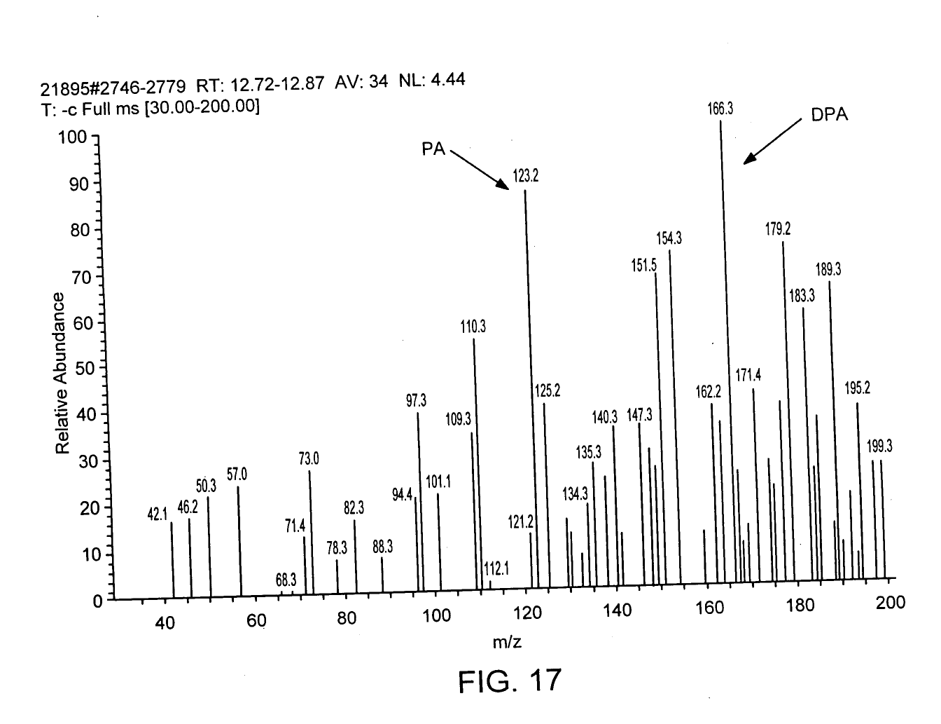

In order to provide a control result for comparison with data obtained using the FAIM spectrometer, a B. subtilis sample was sent to CDS Analytical for analysis. FIG. 17 show the results from pyrolysis-mass spectrometry and indicate that the expected biomarkers can be detected using the pyrolyzer unit discussed above. Specifically, FIG. 17 shows that pyrolysis of the B. subtilis spores produces two unique biomarkers that are unique to endosporulating bacteria. The mass spectrometry data show that the PA peaks at 123 m/z and DPC peaks at 166 m/z. The concentration of the B. subtilis sample was 109 organisms/ml, orders of magnitude above the ultimate detection limit of the FAIM spectrometer system.

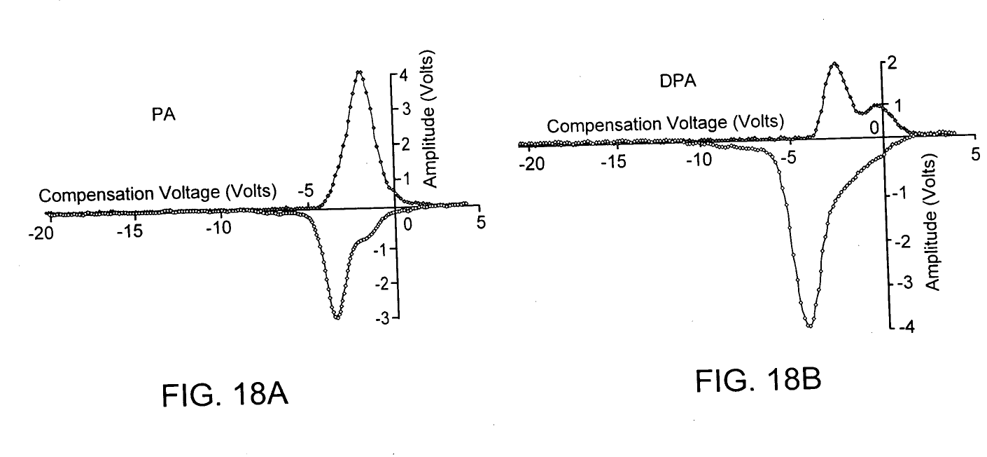

FIG. 18A shows the pyrolysis-FAIM spectrometry for PA and FIG. 18B shows the pyrolysis-FAIM spectrometry for DPA. The spectra were obtained from solid chemical samples pyrolyzed sequentially employing identical FAIM spectrometry operating conditions. Picolinic acid (PA) was pyrolyzed through a temperature excursion from 130° C. to 300° C. at a rate of 20,000° C./s, the interface temperature was held at 130° C. Dipicolinic acid (DPA) was pyrolyzed from 145° C. to 400° C. at 20,000° C./s, the interface temperature was held at 145° C. The FAIM spectra produces both positive and negative unique ion spectra for the PA and DPA spore biomarkers. Both PA and DPA, referring to FIGS. 17A and 17B, respectively, produce positive and negative ion peaks that can be used for identification. In addition DPA produces a secondary positive ion peak, further differentiating its fingerprint pattern. The peak width at half height averages 1.4 V. Even though compound identification is relatively straightforward under these controlled conditions, further optimization is desired to improve resolution of the peaks. It is known that pyrolysis is capable of fully decarboxylating DPA to pyridine. Ideally, controlled and more gradual pyrolysis conditions will lead to loss of only one carboxylic acid group to generate PA, enabling specific identification of the DPA source as bacterial spores.

Secondly, a softer ionization technique such as Matrix-Assisted Laser Desorption/Ionization (MALDI) may also be use. According to MALDI preparation, spore samples are pretreated by, for example, coronal plasma discharge or other chemical means, so that they will yield a high number of biomarkers. The pretreated spores are then complexed with a specific chemical matrix, usually an acid solution. The matrix-spore complex is excited by a laser using an energy level sufficient to excite the matrix but not the pretreated spore itself. The matrix and the spore then split apart, yielding electrostatic charged moieties that are introduced in the mass spectrometer to yield spectral biomarkers.

A second sample ionization and handling approach provides a rich variety of potential biomarker patterns for bioinformatics analysis is the soft-ionization sample introduction method Atmospheric Pressure—Matrix-Assisted Laser Desorption Ionization (AP-MALDI) for solid spore sample introduction into the FAIM spectrometer unit.

For example, a Finnigan AP-MALDI unit can couple with the FAIM spectrometer. Both the pyrolysis, discussed above, and the AP-MALDI ionization methods are performed at atmospheric pressure, thus simplifying the interface technology necessary to introduce the sample into the FAIM spectrometer unit.

B. thuriengiensis (ATCC 10792) is used as a model species for B. anthracis and used to optimize both pyrolysis and AP-MALDI protocols to produce FAIM spectra for solid endospore samples. Briefly, the bacteria is cultured in 100 ml flasks, at 37° C. in a nutrient broth. The cultures are treated under conditions to induce sporulation. To harvest the bacteria, samples of the B. thuringiensis cultures are centrifuged at 10,000×g for 10 minutes. Remaining vegetative cells in the harvested sample are destroyed by treating the sample with lysozyme (50 μg/ml) in 50 mM Tris-HCl (pH 7.2). Bacterial samples are microscopically inspected for the visual presence of spores for each preparation. The spore samples are dried at 70° C. overnight to remove excess water, and 0.1 mg of spore sample is introduced into the pyrolysis or AP-MALDI units connected to the FAIM spectrometer, according to any of the methods described above with respect to introducing the sample to a pyrolysis system or according to AP-MALDI protocols described in the literature.

Control experiments are performed using the B. subtilis strain 168 bacteria (ATCC 23857) prepared in the manner described above with respect to the B. thuriengiensis. Cluster analysis (Correlogic Systems software package) employed in the solid spore sampling aids in specific detection between these two Bacillus species. By comparing these two species, we are able to distinguish between B. anthracis-like markers (from B. thuringiensis) and general non-specific Bacillus endospore markers from the more distantly related B. subtilis. This enables us to determine what is the maximal type of non-specific spore background markers to expect. For example, it enables identification of reference spectra for putative biomarkers such as volatile lipid components of the spore coat and dipicolinic acid (a unique bacterial spore chemical which comprises 5-15% of the mass of spores). The cluster analysis shows a relative number of biomarkers produced using the pyrolysis versus the AP-MALDI sample introduction methods. The introduction protocols are altered to produce more or less unique markers for the B. thuringiensis versus the B. subtilis. This has been shown with specialized matrix preps in MALDI-MS for B. anthracis. Methylation of the spore samples may be employed prior to analysis to increase the number of volatile biomarkers released.

The various growth media for B. thuringiensis may also affect spore preparation and the unique biomarkers that are present. The bacteria and endospore preparation protocols determine how much the biomarkers will shift. It is also possible to determine if there is a way for the B. thuringiensis to appear like B. subtilis or if potential changes in the biomarker patterns simply make the B. thuringiensis appear more distinct from the control B. subtilis patterns. If media/preparation changes produce more distinct spectra, then the background biomarkers from commonly found endospore samples, such as B. subtilis and B. cereus (ATCC 4342) continue to be characterized. If there is a sample preparation method that produces B. thuringiensis biomarkers that are more like B. subtilis, then sample handling protocols are altered and pyrolysis or MALDI technique attempt to liberate more unique biomarkers that are resistant to the effects of media changes. These experiments aid in increased sensitivity of environmental species-specific spore detection.

Both pyrolysis and MALDI introduction techniques are expected to yield a number of useful biomarkers for solid spore samples, and it is possible that sample handling protocols may maximize the number of unique biomarkers produced from these methods.

Collection and concentration methods for air sample introduction of aerosolized B. thuringiensis are optimized to produce FAIM spectral patterns. The threshold of detection is determined and sample handling methods are optimized to replicate reference spectra for B. thuringiensis in response to as little as <100 spores. By optimizing the air sampling protocols for B. thuringiensis, adequate sampling methods for aerosolized samples are ensured. By gauging the sensitivity level and appropriately altering protocols a better resolved FAIM spectra is produced. For example, increasing the power of the FAIM spectrometer ionization source improves the FAIM spectra.

It is possible to differentiate with <100 spores between aerosolized B. thuringiensis and the related non-pathogenic B. subtilis using spectral cluster analysis to identify FAIM spectral biomarker patterns with computational methods similar to those described above for the solid spore samples. This experiment determined that the specificity of biomarkers and cluster analysis still uniquely identifies between Bacillus species even at very low bacteria numbers.

Briefly, both Bacillus samples are aerosolized at known concentrations in an inert N₂ carrier gas. A finite sample of the mixture is introduced into the FAIM spectrometer unit to produce a plot of the predicted number of spores present versus the FAIM spectrometer response amplitude. When less spores are present, there is the possibility for greater spore-to-spore biomarker composition variability. By performing the aerosolized detection experiments with a minimal number of spores, we gauge if the biomarkers chosen to distinguish between species in the solid spore samples are the most resistant to natural biological variation. If detection between species is not achieved, the sample handling protocols are reexamined again to identify more stable biomarkers.

Environmental spore detection is critical to assay potential aerosolized biological weapons deployment. However, spore exposure does not necessarily translate into pathogen infection. In fact, there are critical parameters that determine the effectiveness of aerosolized biological weapons, such as the number of spores delivered and the particle size of the endospores.

In an emergency mass triage setting where an unknown aerosolized pathogen may have been deployed, a rapid and non-invasive method to assay patients for early infection does not currently exist. Furthermore, the symptoms of several common respiratory ailments are easily confused with the early clinical symptoms of class A pathogen infection. Accordingly, there is need for a rapid detection method that distinguishes between pulmonary infection with aerosolized bioagents and other clinical conditions.

Disease resulting from exposure to B. anthracis spores can be categorized into cutaneous, gastrointestinal and inhalational anthrax. Of these three forms, inhalational anthrax presents the most severe clinical symptoms, usually resulting in a high mortality rate if not treated early in disease progression.

Presently, if exposure to aerosolized B. anthracis spores is suspected, patients are treated by oral or intravenously administered antibiotics, such as ciprofloxacin or doxycycline, as indicated by clinical conditions. The clinical diagnosis of anthrax is confirmed by direct visualization or culturing of the anthrax bacilli. Fresh smears of various body fluids are stained with polychrome methylene blue and examined for the characteristic square-ended, blue-black bacilli surrounded by a pink capsule. Late identification of exposure and infection greatly increases patient mortality rates. Gastrointestinal and pulmonary anthrax infections are difficult to identify before the final phases of disease, and therefore carry a high mortality. Often patients will be given prophylactic drug treatment presuming they are infected after confirmed or suspected exposure. However, this preemptive antibiotic use can result in false-negative evidence for exposure, as well as significant drug side effects. One clinical detection method under investigation uses DNA microarray technology. Many potential biomarkers will be detectable in clinical breath samples.

Reference biomarkers and biomarker patterns are identified for use in correlating patient breath samples. In some cases patient breath samples are correlated with known reference biomarker patterns. Also, breath samples may be spiked with a known biomarker and correlated to provide reference biomarker patterns. In one embodiment, metabolic bacterial reference biomarkers are identified by collecting gaseous headspace samples above vegetative liquid cell cultures. The headspace samples are correlated with biomarker patterns in patient breath samples and in spiked breath samples.

For example, the reference biomarkers from early vegetative B. thuringiensis (ATCC 10792), B. anthracis, and type III Streptococcus pneumoniae (ATCC 10813) in liquid culture may also be identified. By comparing the B. thuringiensis vegetative markers to the solid spore biomarkers previously identified, a correlation between spore and potential vegetative markers is examined. The identified vegetative S. pneumoniae markers serve as controls for biomarkers from bacterial pneumonia patient breath samples. Putative candidate biomarkers include 18-hexadiamic acid, a volatile lipid produced only in germinating B. anthracis cells.

Bacterial cultures are grown in 100 ml Erlenmeyerflasks optimized for headspace sampling. Bacterial cultures are maintained at log phase growth in 100 ml Erlenmeyer flasks optimized for anaerobic cultures and headspace sampling. Gaseous headspace samples are drawn off the liquid cultures in 4 hour intervals for the first 12 hours after culture inoculation, with a control measurement taken at time 0 hour. This sampling method identifies vegetative markers that may change with time as the bacteria proliferate. If there are radical differences in biomarkers present at time 0 hour and 4 hours, then the testing procedure is revised and samples are taken from the headspace gases every hour starting at inoculation. Similar techniques may be employed to identify reference biomarkers to be employed in accordance with the invention.

The B. anthracis vegetative biomarker testing is performed in a functional Containment Core facility. Two B. anthracis strains are utilized for this portion of the experiment, each strain containing one of the virulence plasmids: pXO1 (Sterne strain 7702) and pXO2 (strain 9131). FAIM spectrometer biomarkers are identified from each of these strains. By comparing the patterns, a greater number of biomarkers applicable to early anthrax infection in clinical breath samples are identified. Biomarkers common to both strains are first identified. Thereafter, unique biomarkers are individually identified for each strain. The biomarkers unique to the 9131 strain are due to the presence of the bacterial capsule and the biomarkers unique to the 7702 strain are related to the plasmid encoding anthrax toxins. The relative number of biomarkers common to these 2 species is proportional to the number of genes in the B. anthracis bacterial chromosome. Similarly, the number of unique biomarkers between these two attenuated strains is proportional to the number of genes on each of the plasmids.

Specific FAIM spectral biomarkers correlated with clinical pulmonary infectious diseases are identified in samples of patient breath exhalate, with specific attention to clinical conditions such as pneumonia and influenza that are easily confused with early symptoms of infection with category A pathogens.

FAIM spectrometry provides a mass-producible monitor for pathogen detection that may be employed, for example, for environmental monitoring of bacterial spores. FAIM spectrometry may be employed to investigate biomarkers for other Category A-C pathogens and to chemically fingerprint these species. As discussed above, FAIM spectrometry may be employed for breath analysis and for, for example, the early detection and diagnosis of disease.

While the invention has been shown and described with reference to specific embodiments various combinations of elements, it should be understood by those skilled in the art that various changes in form and detail may be made therein without departing from the spirit and scope of the invention.

The terms and expressions employed herein are used as terms of description and not of limitation, and there is no intention, in the use of such terms and expressions, of excluding any equivalents of the features shown and described or portions thereof. Having thus described certain embodiments of the present invention, various alterations, modifications, and improvements will be apparent to those of ordinary skill. Such alterations, modifications, and improvements are within the spirit and scope of the invention, and the foregoing description of certain embodiments is not exhaustive or limiting.

10. Experimental Results

Experiment Number 1

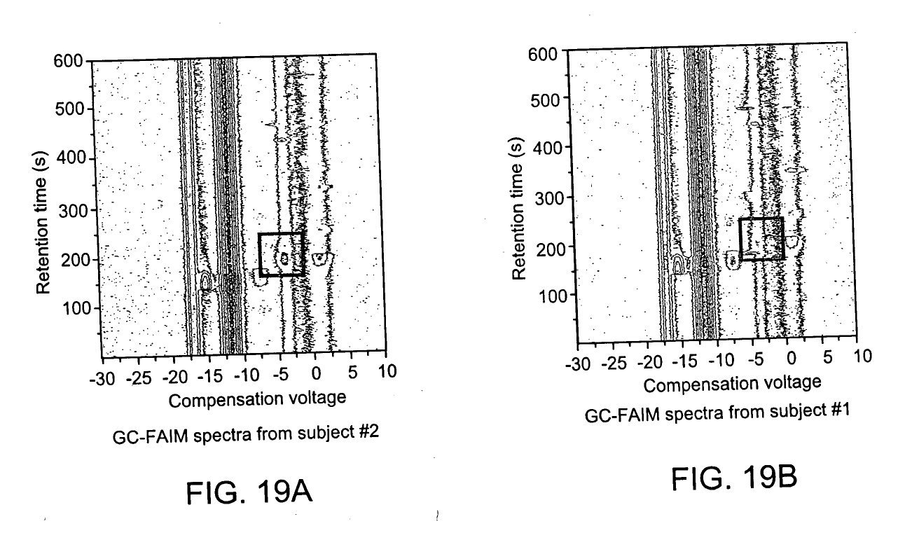

Preliminary breath analysis experiments were performed on two subjects with the FAIM spectrometer and the spectra results are provided at FIG. 19A for subject #2 and at FIG. 19B for subject #1. In these experiments, sample collection involved collecting a breath sample directly onto a SPME fiber assembly. The SPME fiber was placed in proximity to the mouth of the patient (subject #1) and the sample collected for 2 minutes. The SPME breath sample collection method was performed on a second patient (subject #2). For each sample, after sample collection, the SPME assembly was inserted into a Gas Chromatography (GC) injector port which was held at 120° C. and desorbed the breath sample from the fiber into the GC column. The FAIM spectrometer was attached at the detector port of the GC. The resultant GC-FAIM spectrometer plot shows the chromatographic retention time on the y-axis and the FAIM spectrometer compensation voltage plotted on the x-axis. Although the experimental data is only for a limited sample, the data indicates that the FAIM spectrometer provides additional information to simplify and assist in the analysis of a human breath sample. The spectra from subject #1 and subject #2 are very similar except for the peak at a compensation of about −3 volts for the sample from subject #2. Using the GC alone, the presence of the different compound in the sample from subject #2, indicated by the peak, would not be evident.

Experiment Number 2

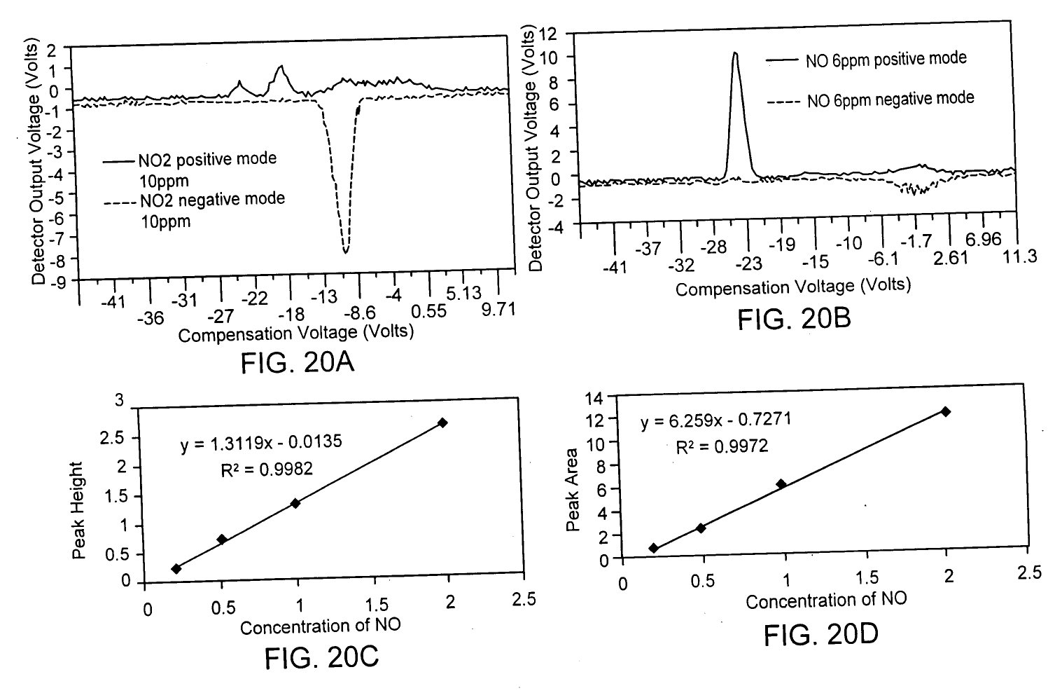

FAIM spectrometer detection is sensitive enough for both NO and NO₂ in the low ppm and mid ppb range, as shown in FIG. 20A-20D. A micromachined FAIM spectrometer was used, together with a UV ionization source, to collect reference spectra for both NO and NO₂. The experiment was conducted at room temperature using high purity nitrogen (99.9995%) as drift gas. A mass flow controller precisely diluted both NO and NO₂ to the desired concentration of 1500 ppm. FIG. 20A shows FAIM spectrometer spectra where the x-axis is compensation voltage and the y-axis is detector output voltage, including peak responses for low ppm NO₂ concentrations, specifically at a 10 ppm concentration. FIG. 20B shows FAIM spectrometer spectra where the x-axis is compensation voltage and the y-axis is detector output voltage, including peak responses for low ppm NO concentrations, specifically at a concentration of 6 ppm. FIG. 20C shows the resultant spectra plot with concentration of NO on the x-axis and peak height on the y-axis. The FAIM spectra were collected without averaging, and are linear as a function of peak height over a wide range of concentrations, down to 200 ppb NO concentrations. FIG. 20D shows the resultant spectra plot with concentration of NO on the x-axis and peak area on the y-axis. The FAIM spectra were collected without averaging, and are linear as a function of peak area over a wide range of concentrations, down to 200 ppb NO concentrations. The FAIM spectra sensitivity to NO is applicable for breath analysis, for example, as an early indicator of macrophage activation in pulmonary infection.

Experiment Number 3

FAIM spectrometry may be employed to identify spectral biomarker patterns, other than elevated NO levels, in a variety of pulmonary infections that are easily confused with early infection of category A pathogens. Novel biomarkers are identified in breath exhalate samples from clinically diagnosed pneumonia patients via cluster pattern analysis. Species-specific spectral patterns from clinically diagnosed (by Infectious Diseases Society of America guidelines) pulmonary infections, such as Streptococcus pneumoniae, Staphylococcus aureus, Haemophilus influenzae and Mycoplasma pneumoniae are measured and determined. Cluster pattern analysis identifies specific biomarkers in breath exhalate samples for clinically confirmed viral influenza and viral pneumonia patients. Finally, cluster pattern analysis identifies specific biomarkers in breath exhalate samples for clinically confirmed anaerobic bacterial infection patients.

Aerosolization with subsequent inhalation is seen as the major vehicle for delivery of most bioweapons agents. Therefore, the exhaled breath will likely contain early indicators of infection from these agents. For instance, the transformation of spores to the vegetative state produces a range of biomarkers specific to the particular agent. The high sensitivity of the FAIM spectrometer enables detection of such byproducts from a breath sample. The pathogenicity of B. anthracis depends on two virulence factors: a poly-y-D-glutamic acid polypeptide capsule, which protects it from phagocytosis by the host, and toxins produced in the log phase of growth. These toxins consist of three proteins: protective antigen (PA) (82.7 kDa), lethal factor (LF) (90.2 kDa), and edema factor (EF) (88.9 kDa). These B. anthracis products create detectable volatile chemical signatures that are detectable in breath samples. It is extremely unlikely that patients would shed spores through exhaled breath after exposure. This is highlighted by the lack of secondary cases or “spread” of inhalational anthrax between patients.

1. A system for breath analysis comprising:
providing a breath sample;
providing an asymmetric field ion mobility spectrometer comprising:
an ionization source for ionizing the breath sample and creating ions;
an analytical gap enclosed by a housing;
an ion filter disposed in the analytical gap downstream from the ionization source, and including electrodes on an inside surface of the housing for creating an asymmetric electric field to filter the ions;
an ion flow generator including electrodes proximate but insulated with respect to the ion filter electrodes for creating an electric field transverse to the asymmetric electric field for propelling ions through the asymmetric electric field; and
an ion detector for sensing ions not filtered by the ion filter.

2. The system of claim 1 wherein providing a breath sample comprises:
providing a constant rate of breath expiration.

3. The system of claim 1 further comprising:
introducing the breath sample into the spectrometer.

4. The system of claim 3 wherein the breath sample is introduced at a constant rate.

5. The system of claim 3 wherein a fixed volume of the breath sample is introduced into the spectrometer.

6. The system of claim 3 further comprising a pressure source for introducing the breath sample into the spectrometer.

7. The system of claim 3 wherein the breath sample is exhaled into the spectrometer.

8. The system of claim 7 further comprising:
providing a-channel adapted to introduce the exhaled breath sample into the spectrometer.

9. The system of claim 8 wherein the channel comprises a mouthpiece.

10. The system of claim 3 wherein the breath sample is contained in a collection vessel.

11. The system of claim 10 further comprising:
providing a conduit adapted to introduce the breath sample contained in the collection vessel into the spectrometer.

12. The system of claim 1 wherein providing a breath sample comprises:
providing a signal to a user that the breath sample rate is constant.

13. The system of claim 1 further comprising providing intermediate analytical separation of the breath sample prior to introducing the breath sample to the spectrometer.

14. The system of claim 1 wherein providing a breath sample comprises:
breathing according to a standard protocol prior to providing the breath sample.

15. The system of claim 1 wherein the breath sample is taken from a patient suspected to have at least pulmonary infection, metabolic disease, chronic progressive degenerative pulmonary disease, lung cancer, or organ dysfunction.

16. The system of claim 1 wherein the breath sample is taken from a patient receiving a course of drug therapy.

17. The system of claim 1 wherein the breath sample is taken from a patient suspected to have been exposed to industrial chemicals.

18. The system of claim 1 wherein the ion detector is adapted to sense ions indicative of nitric oxide level.

19. The system of claim 1 wherein the ion detector is adapted to sense ions indicative of at least pulmonary infection, pulmonary inflammation, metabolic disease, chronic progressive degenerative pulmonary disease, lung cancer, organ dysfunction, or industrial chemical exposure.

20. The system of claim 1 wherein the ion detector is adapted to sense ions of biomarkers indicative of response to drug therapy.

21. The system of claim 1 wherein the ion detector is adapted to sense ions indicative of at least bacterial infection, viral infection, fungal infection, yeast infection, infectious disease agents, response to biowarfare agents, or emerging infectious disease agents.

22. The system of claim 1 wherein the spectrometer is hand held.

23. The system of claim 1 wherein the spectrometer is adapted to have an independent power supply.

24. The system of claim 1 wherein the spectrometer is adapted to be fieldable.

25. The system of claim 1 further comprising:
providing a data collector to collect the ion sensed by the ion detector; and
evaluating the collected data for a pattern.

26. The system of claim 25 wherein the data collector is a personal data assistant.

27. The system of claim 25 wherein the data collector is disposed on the housing of the spectrometer.

28. An asymmetric field ion mobility spectrometer for breath analysis comprising:
an ionization source for ionizing a breath sample and creating ions;
an analytical gap enclosed by a housing;
an ion filter disposed in the analytical gap downstream from the ionization source, and including electrodes on an inside surface of the housing for creating an asymmetric electric field to filter the ions;
an ion flow generator including electrodes proximate but insulated with respect to the ion filter electrodes for creating an electric field transverse to the asymmetric electric field for propelling ions through the asymmetric electric field; and
an ion detector for sensing ions not filtered by the ion filter.

29. An asymmetric field ion mobility spectrometer for breath analysis comprising:
an ionization source for ionizing a breath sample and creating ions;
an analytical gap;
an ion filter disposed in the analytical gap downstream from the ionization source, and including a pair of spaced electrodes for creating an asymmetric electric field to filter the ions;
an ion flow generator including a plurality of spaced discrete electrodes insulated from the pair of spaced electrodes for creating an electric field transverse to the asymmetric electric field for propelling ions through the asymmetric electric field; and
an ion detector for sensing ions not filtered by the ion filter.

30. An asymmetric field ion mobility apparatus for identification of ion species in a breath sample, the apparatus comprising:
an ionization source for ionizing a breath sample and creating ions;
an ion filter disposed in a flow path, said flow path having a longitudinal axis for the flow of ions, said filter supplying an asymmetric filter field transverse to said longitudinal axis, said asymmetric filter field being compensated;
an ion flow generator for longitudinally propelling ions along said flow path in said compensated asymmetric filter field; and
the ion filter passing a species of said propelled ions, said species having a set of characteristics correlated with said compensated asymmetric filter field, said correlation facilitating identification of said species.
